(19) United States
(12) Patent Application Publication
(10) Pub. No.: US 2022/0331257 A1
(43) Pub. Date: Oct. 20, 2022

(54) LIQUID PHARMACEUTICAL COMPOSITIONS WITH STABLE DRUG RELEASE PROFILES

(71) Applicant: Avadel Legacy Pharmaceuticals, LLC, Chesterfield, MO (US)

(72) Inventors: CHARLES SANSON, Chesterfield, MO (US); FARAH MARZOUK, Chesterfield, MO (US); GAUTHIER POULIQUEN, Chesterfield, MO (US)

(21) Appl. No.: 17/435,000

(22) PCT Filed: Feb. 28, 2020
(86) PCT No.: PCT/US20/20408

§ 371 Date: Aug. 30, 2021

Related U.S. Application Data
(60) Provisional application No. 62/812,705, filed on Mar. 1, 2019.

(51) Int. Cl. A61K 9/50; A61K 31/155; A61K 31/167; A61K 31/09

(52) U.S. Cl. CPC A61K 9/5026; A61K 9/5063; A61K 31/155; A61K 31/167; A61K 31/09

(57) ABSTRACT

The present disclosure provides compositions having drug-containing particles suspended in a continuous phase that is not saturated by the drug that are stable after being stored as a suspension for more than 1 month at 30° C.

### CROSS-REFERENCE TO RELATED APPLICATIONS

This application claims priority to U.S. Provisional Application No. 62/812,705, filed Mar. 1, 2019.

### FIELD

The present disclosure generally relates to liquid pharmaceutical compositions with stable drug release profiles.

### BACKGROUND

Drug-containing particles dispersed in a continuous phase not saturated by the drug and stored as a suspension may release the drug into the continuous phase after storage for an extended period of time. This can affect the dosage as it increases the amount of drug in immediate-release form rather than in the particles, which may be gastro-soluble, sustained release, or delayed release particles.

Therefore, there is a need for drug-containing particles and suspensions thereof in a continuous phase that is not saturated by the drug that are stable when stored for weeks or months at room temperature.

### SUMMARY

In an aspect, the present disclosure encompasses a particle including a drug-containing core and an outer layer covering the core. The outer layer may include a hydrophobic compound that is lipidic with a melting temperature between about 50° C. and about 90° C. and a gastro-soluble polymer comprising a minimum of 50% molar ratio of methyl methacrylate and a maximum of 50% molar ratio of an amino alkyl methacrylate. The gastro-soluble polymer may exhibit a glass-transition temperature (Tg) of greater than 50° C. and an overall molecular weight (Mw) greater than 50 000 g/mol. The hydrophobic compound and gastro-soluble polymer may be present in a weight ratio of at least 2.3.

In another aspect, the present disclosure encompasses a pharmaceutical composition including a plurality of particles dispersed in a continuous phase, forming a suspension. The continuous phase may be (a) not saturated by the drug contained in the particles, (b) include at least one osmotic agent, and (c) have a pH that is above the gastro-soluble polymer's pKa. The osmolality of the continuous phase may be (i) higher than saturated solution of the drug in water and (ii) higher than a minimum value of 1300 mOsm/kg. In an aspect, the suspension may have a stable in vitro dissolution profile after storage for at least one month at about 30° C. In another aspect, the amount of drug in the continuous phase of the suspension is reduced by a factor of 10 compared to the saturation of the continuous phase by the drug.

In yet another aspect, the present disclosure encompasses a pharmaceutical composition including a plurality of particles dispersed in a continuous phase, forming a suspension, where (a) each particle includes a drug-containing core and an outer layer covering the core; (b) the continuous phase is not saturated with a drug and comprises at least one osmotic agent; (c) the osmolality of the continuous phase is higher than the osmolality of a saturated solution of the drug in water; and (d) after storage of the composition in suspension for at least one month at about 30° C., the composition has a stable in vitro dissolution profile. The drug may have a coating/water partitioning coefficient K that is less than one. The outer layer may include (i) up to 20 wt % of one or more water-soluble polymers and (ii) at least about 80 wt % of a mixture including a cellulosic derivative insoluble in the gastrointestinal tract and a plasticizer.

In an aspect, the present disclosure encompasses a delayed-release pharmaceutical composition including a plurality of particles dispersed in a continuous phase, forming a suspension, where (a) each particle consists essentially of a drug-containing core and an outer layer covering the core; (b) the continuous phase (i) is not saturated with a drug, (ii) comprises at least one osmotic agent, and (iii) has a pH below 3.5; (c) the osmolality of the continuous phase is higher than the osmolality of a saturated solution of the drug in water and greater than 1300 mOsm/kg; and (d) after storage of the composition in suspension for at least one month at about 30° C., the composition has a stable in vitro dissolution profile. In an aspect, the outer layer includes one or more hydrophobic compounds that are crystalline in the solid state and one or more polymers carrying groups that are ionized at neutral pH. The weight ratio of the hydrophobic compound(s) to the polymer(s) carrying groups that are ionized at neutral pH may be greater than 1.5.

Other aspects and iterations of the invention are described more thoroughly below.

### BRIEF DESCRIPTION OF THE FIGURES

The application file contains at least one photograph executed in color. Copies of this patent application publication with color photographs will be provided by the Office upon request and payment of the necessary fee.

FIG. 1 graphically depicts the results of in vitro dissolution tests (USP II, 37° C., PB pH 6.8). The samples tested are dry particles (blue diamonds), and a suspension stored for 1 week at 40° C. (red squares). Further details are found in Experiment 1 of Example 7. The percent of drug released into the dissolution medium is graphed on the y-axis versus time (hours, x-axis). The in vitro dissolution profile of the suspension after storage differs from the in vitro dissolution profile of the initial solution and the dry particles.

FIG. 2 graphically depicts the results of in vitro dissolution tests (USP II, 37° C., PB pH 6.8). The samples tested are dry particles (blue stars) and a suspension thereof stored for 1 week at 40° C. (red squares). Further details are found in Experiment 2 of Example 7. The percent of drug released into the dissolution medium is graphed on the y-axis versus time (hours, x-axis). The in vitro dissolution profile of the suspension after storage differs from the in vitro initial dissolution profile of the dry particles.

FIG. 3 graphically depicts the results of in vitro dissolution tests (USP II, 37° C., 0.1N HCl pH 1.1). The samples tested are dry particles (red circles), a suspension thereof stored for 1 week at 30° C. (blue circles). Further details are found in Experiment 3 of Example 7. The percent of drug released into the dissolution medium is graphed on the y-axis versus time (hours, x-axis). The in vitro dissolution profiles of the suspensions after storage differ from the in vitro initial dissolution profile.

FIG. 4 graphically depicts the results of in vitro dissolution tests (USP II, 37° C., PB pH 6.8). The samples tested are dry particles (blue diamonds), a suspension thereof stored for 1 week at 40° C. (green triangles). Further details are found in Experiment 4 of Example 7. The percent of drug released into the dissolution medium is graphed on the y-axis versus time (hours, x-axis). The in vitro dissolution profile of the suspension after storage differs from the in vitro dissolution profile of the initial solution and the dry particles.

FIG. 5 graphically depicts the results of an in vitro dissolution tests (USP II, 37° C., PB pH 6.8). The samples tested are dry particles (orange circles), and a suspension thereof stored for 1 week at 30° C. (red circles). Further details are found in Experiment 5 of Example 7. The percent of drug released into the dissolution medium is graphed on the y-axis versus time (hours, x-axis). The in vitro dissolution profile of the suspension after storage differs from the initial dissolution profile of the dry particles.

FIG. 6A and FIG. 6B show the stable behavior of the APAP particles of Example 1 in suspension. The particles have a KOLLICOAT® Smartseal 30 D/LUBRITAB® (30/70) coating (CR=30 wt %). KOLLICOAT® Smartseal 30 D is an aqueous dispersion of a co-polymer comprising methyl methacrylate (MMA) and diethylaminoethyl methacrylate (DEAEMA) in a ratio of about 6:4—it also contains macrogol cetostearylether 20 (˜0.6%) and sodium lauryl sulfate (˜0.8%) as stabilizers and has a solids content of 30%. The continuous phase of the suspension comprises 60 wt % maltitol and is buffered to pH>7 with KH2PO4. FIG. 6A graphically depicts the solubilized drug fraction in the continuous phase of the suspension (y-axis), as assayed by HPLC, after storage for various amounts of time (x-axis) at 30° C. Also indicated on the graph is the amount of drug required to reach saturation (dashed orange line). The solubilized content of APAP in the continuous phase of the suspension is reduced by a factor of 10 compared to the saturation of the continuous phase by the drug after six months of storage. FIG. 6B graphically depicts the results of in vitro dissolution tests (USP II, 37° C., 0.1N HCl) of the initial suspension (blue circles) and the suspension after storage for 3 months at 30° C. (orange circles). The percent of drug released into the dissolution medium is graphed on the y-axis versus time (hours, x-axis). While no leaching of the API is observed in the continuous phase over time, the immediate release profile is maintained over time once dissolution is performed. To is performed immediately (<30 min) after the suspension is made.

FIG. 7 graphically depicts the solubilized APAP fraction in the continuous phase of a suspension (y-axis), as assayed by HPLC, after storage for various amounts of time (x-axis) at 30° C. Also indicated on the graph is the amount of APAP required to reach saturation (dashed orange line). The samples tested are suspensions of particles with a KOLLICOAT® Smartseal 30 D/LUBRITAB® (30/70) coating (blue circles) or a EUDRAGIT® E/LUBRITAB® (30/70) coating (green circles). Both suspensions contain 60 wt % maltitol as an osmotic agent and is buffered to pH≥7 with KH2PO4. FIG. 7 shows use of EUDRAGIT® E in the outer layer does not impart the same stability characteristics as use of KOLLICOAT® Smartseal 30 D.

FIG. 8A and FIG. 8B show the stable behavior of the Metformin HCl (1,1-dimethylbiguanide hydrochloride) particles of Example 2 in suspension. The particles have a KOLLICOAT® Smartseal 30 D/LUBRITAB® (20/80) coating (CR=30 wt %). The continuous phase of the suspension comprises 60 wt % maltitol and is buffered to pH≥7 with KH2PO4. FIG. 8A graphically depicts the solubilized drug fraction in the continuous phase of the suspension (y-axis), as assayed by HPLC, after storage for various amounts of time (x-axis) at 30° C. The solubilized content of Metformin HCl in the continuous phase of the suspension is reduced by a factor of 10 compared to the saturation of the continuous phase by the drug after six months of storage. FIG. 8B graphically depicts the results of in vitro dissolution tests (USP II, 37° C., 0.1 N HCl) of the initial suspension (blue circles) and the suspension after storage for 6 months at 30° C. (orange circles). The percent of drug released into the dissolution medium is graphed on the y-axis versus time (hours, x-axis). The in vitro dissolution profile of the suspension after storage does not substantially differ from its initial dissolution profile. While no leaching of the API is observed in the continuous phase overtime, the immediate release profile is maintained over time once dissolution is performed. To is performed immediately (<30 min) after the suspension is made.

FIG. 9A and FIG. 9B show the stable behavior of particles with an immediate release coating on a core of guaifenesin of Example 3 in suspension. The particles have a KOLLICOAT® Smartseal 30 D/LUBRITAB® (30/70) coating (CR=30 wt %). The continuous phase of the suspension comprises 60 wt % maltitol and is buffered to pH≥7 with KH2PO4. FIG. 9A graphically depicts the solubilized drug fraction in the continuous phase of the suspension (y-axis), as assayed by HPLC, after storage for various amounts of time (x-axis) at 30° C. The solubilized content of guaifenesin in the continuous phase of the suspension is 0.5 mg/g after one month of storage and 1.1 mg/g after 2 months of storage. FIG. 9A shows that the free fraction of guaifenesin after 2 months of storage is reduced by a factor of 10 compared to the saturation of the continuous phase by the drug. FIG. 9B graphically depicts the results of in vitro dissolution tests (USP II, 37° C., 0.1N HCl) of the initial suspension (blue circles) and the suspension after storage for 3 months (orange circles) at 30° C. The percent of drug released into the dissolution medium is graphed on the y-axis versus time (hours, x-axis). The in vitro dissolution profiles of the suspensions after storage do not substantially differ from its initial dissolution profile. While no leaching of the API is observed in the continuous phase over time, the immediate release profile is maintained over time once dissolution is performed. To is performed immediately (<30 min) after the suspension is made.

FIG. 10A and FIG. 10B show the stable behavior of extended release guaifenesin particles with an immediate release overcoat of Example 3 in suspension. The particles have a KOLLICOAT® Smartseal 30 D/LUBRITAB® (20/80) over-coating (CR=30 wt %). The continuous phase of the suspension comprises 60 wt % maltitol and is buffered to pH≥7 with KH2PO4. FIG. 10A graphically depicts the solubilized drug fraction in the continuous phase of the suspension (y-axis), as assayed by HPLC, after storage for various amounts of time (x-axis) at 30° C. The solubilized content of guaifenesin in the continuous phase of the suspension is 0.13 mg/g after one month of storage. FIG. 10A shows the free fraction of guaifenesin at 1 month is reduced by a factor of 10 compared to the saturation of the continuous phase by the drug. FIG. 10B graphically depicts the results of in vitro dissolution tests (USP II, 37° C., 0.1N HCl) of the initial suspension (blue circles) and the suspension after storage for 1 month (orange circles) at 30° C. The percent of drug released into the dissolution medium is graphed on the y-axis versus time (hours, x-axis). FIGS. 10A and 10B show that the immediate release overcoat does not modify the sustained release profile of the guaifenesin particle. T₀ is performed immediately (<30 min) after the suspension is made.

FIG. 11A and FIG. 11B show the stable behavior of extended release APAP particles of Example 4 in suspension. The particles have a KOLLICOAT® Smartseal 30 D/LUBRITAB® (20/80) over-coating (CR=30 wt %). The continuous phase of the suspension comprises 60 wt % maltitol and is buffered to pH≥7 with KH2PO4. FIG. 11A graphically depicts the solubilized drug fraction in the continuous phase of the suspension (y-axis), as assayed by HPLC, after storage for various amounts of time (x-axis) at 30° C. The solubilized content of APAP in the continuous phase of the suspension is 0.5 mg/g after one month of storage. FIG. 11A shows that the free fraction of APAP at 1 month is reduced by a factor of 10 compared to the saturation of the continuous phase by the drug. FIG. 11B graphically depicts the results of in vitro dissolution tests (USP II, 37° C., 0.1N HCl) of the initial suspension (blue circles) and the suspension after storage for 1 month (orange circles) at 30° C. The percent of drug released into the dissolution medium is graphed on the y-axis versus time (hours, x-axis). The in vitro dissolution profiles of the suspensions after storage do not substantially differ from its initial dissolution profile. To is performed immediately (<30 min) after the suspension is made.

FIG. 12 graphically depicts the results of in vitro dissolution tests (USP II, 37° C., 50 mM PB pH 6.8). The samples tested are dry Metformin HCl particles with an EC/PVP/CO (80/10/10) coating (CR=45 wt %, blue diamonds), a suspension thereof stored for 1 week at 40° C. (orange circles), a suspension stored for 1 month at 40° C. (green triangles), and a suspension stored for 1 year at 40° C. (purple x). The particles and suspensions are described in further detail in Example 6. The percent of drug released into the dissolution medium is graphed on the y-axis versus time (hours, x-axis). The in vitro dissolution profiles of the suspensions after storage do not substantially differ from the initial dissolution profile of the dry particles. The stored suspensions have a stable in vitro dissolution profile. FIG. 12 also shows the importance of osmotic control (blue stars).

FIG. 13A and FIG. 13B graphically depict the results of in vitro dissolution tests (USP II, 37° C., PB pH 6.8). In FIG. 13A, the samples tested are dry particles of Metformin HCl with an EC/PVP/CO (80/10/10) coating (CR=15 wt %, blue diamonds) and a suspension thereof stored for 1 month at 30° C. (purple x). In FIG. 13B, the samples tested are dry particles with an EC/PVP/CO (80/10/10) coating (CR=20 wt %, red squares) and a suspension thereof stored for 1 month at 30° C. (green diamonds). The particles and suspensions are described in further detail in Example 6. The percent of drug released into the dissolution medium is graphed on the y-axis versus time (hours, x-axis). The in vitro dissolution profiles of the suspensions after storage do not substantially differ from the initial dissolution profiles of the dry particles.

FIG. 14 graphically depicts the effect of the PVP content on the stability of a particle of the present disclosure. The samples tested are dry particles of Metformin HCl with an EC/PVP/CO coating (CR=30 wt %) containing 10 wt % CO and varying amounts of EC and PVP, or suspensions thereof stored for 1 month at 30° C. The particles and suspensions are described in further detail in Example 6. Graphed on the y-axis is the amount of drug released (wt %) into the continuous phase of the suspension after storage (green triangle) The x-axis is the amount of PVP in the particle's coating (wt %).

FIG. 15 graphically depicts the results of in vitro dissolution tests (USP II, 37° C., PB pH 6.8). The samples tested are dry particles of Metformin HCl with an EC/PVP/CO (70/20/10) coating (CR=30 wt %, green triangles), dry particles with EC/PVP/CO (80/10/10) coating (CR=30 wt %, blue X), and suspensions thereof stored for 6 months at 30° C. (purple diamonds and orange triangle, respectively). The particles and suspensions are described in further detail in Example 6. The percent of drug released into the dissolution medium is graphed on the y-axis versus time (hours, x-axis). The in vitro dissolution profiles of the suspensions after storage do not substantially differ from the initial dissolution profiles of the dry particles.

FIG. 16A and FIG. 16B show that the suspension remains stable when the osmolality of the continuous phase reaches osmolality of a saturated solution of the drug contained in the particles. FIG. 16A and FIG. 16B graphically depict the effect of a composition's osmolality ratio on the stability of a particle of the present disclosure. The samples tested are suspensions comprising particles of Metformin HCl with an EC/PVP/CO (80/10/10) coating, at coating ratio of 30 wt %. The continuous phases of the suspensions comprise various amounts of maltitol as an osmotic agent and therefore each composition has a different external osmolality. On FIG. 16B, after storage of the suspensions for 1 month at 30° C., the amount of drug in the continuous phase was determined. This amount is graphed on the y-axis versus the osmolality of the external phase. The osmolality of the internal phase of the compositions, which is the same for each composition, is also indicated. FIG. 16A graphically depicts the effect of osmolality of the continuous phase on the stability of the in vitro dissolution profile of the particles after 1 month stability in suspension. The percent of drug released into the dissolution medium is graphed on the y-axis versus time (hours, x-axis). The in vitro dissolution profiles of the suspensions after storage do not substantially differ from the initial dissolution profiles of the dry particles when the osmolality ratio is higher than 1. The free fraction in FIG. 16B is from the t_{15 min} dissolution test from FIG. 16A.

FIG. 17 graphically depicts the results of an in vitro dissolution tests (USP II, 37° C., PB pH 6.8). The samples tested are dry particles of Metformin HCl with an EC/Plasdone S-630/CO (80/10/10) coating (blue diamonds) and a suspension thereof stored for 1 month at 40° C. (red squares). The particles and suspensions are described in further detail in Example 6. The percent of drug released into the dissolution medium is graphed on the y-axis versus time (hours, x-axis). The in vitro dissolution profiles of the suspensions after storage do not substantially differ from the initial dissolution profiles of the dry particles.

FIG. 18 is a chart depicting the log K_{film/water} coefficient for a variety of drugs based on an EC/PVP/CO (80/101/10) film.

FIG. 19 graphically depicts the results of an in vitro dissolution test (USP II, 37° C., PB pH 6.8). The samples tested are dry particles of chlorpheniramine.maleate with an EC/PVP/CO (72/20/8) coating (CR=30 wt %; purple X) and a suspension without osmotic agent thereof stored for 1 week at 40° C. (red squares) and a suspension with osmotic agent thereof stored for 3 months at 40° C. (green triangles). The particles and suspensions are described in further detail in Example 5. The percent of drug released into the dissolution medium is graphed on the y-axis versus time (hours, x-axis). The in vitro dissolution profile of the suspension with osmotic agent after storage does not substantially differ from the in vitro initial dissolution profile of the dry particles.

FIG. 20 graphically depicts the results of an in vitro dissolution test (USP II, 37° C., PB pH 6.8). The samples tested are dry particles of Diphenhydramine HCl with an EC/PVP/CO (80/10/10) coating (CR=30 wt %; blue diamonds) and a suspension without osmotic agent thereof stored for 1 week at 40° C. (blue x) and a suspension with osmotic agent thereof stored for 2 months at 40° C. (red squares). The particles and suspensions are described in further detail in Example 5. The percent of drug released into the dissolution medium is graphed on the y-axis versus time (hours, x-axis). The in vitro dissolution profile of the suspension with osmotic agent after storage does not substantially differ from the in vitro initial dissolution profile of the dry particles.

FIG. 21 graphically depicts the results of in vitro dissolution tests (USP II, 37° C., 0.1N HCl). The samples tested are dry particles of APAP with a EUDRAGIT® S100/LUBRITAB® (40/60) coating (CR=25 wt %, purple x) and suspensions thereof stored for 6 months at 30° C. The suspensions have a continuous phase comprising either 60 wt % sorbitol (light blue squares) or 60 wt % maltitol (dark blue diamonds) as the osmotic agent and adjusted to pH=3. The particles and suspensions are described in further detail in Example 8. The percent of drug released into the dissolution medium is graphed on the y-axis versus time (hours, x-axis). The in vitro dissolution profiles of the suspensions after storage do not substantially differ from the in vitro initial dissolution profile of the dry particles.

FIG. 22 graphically depicts the results of in vitro dissolution tests (USP II, 37° C., 0.1N HCl or 0.1N HCl for 3 hours then PB pH 7). The samples tested are suspensions of 60 wt % sorbitol (pH=3) stored for 3 months at 30° C. The APAP particles in the suspension have a EUDRAGIT® S100/LUBRITAB® (40/60) coating (CR=25 wt %). The in vitro dissolution tests occurred at 0.1N HCl for the duration of the test (blue diamonds) or at 0.1N HCl for 3 hours and then at pH 7 (red squares). The particles and suspensions are described in further detail in Example 8. The percent of drug released into the dissolution medium is graphed on the y-axis versus time (hours, x-axis).

FIG. 23 graphically depicts the results of in vitro dissolution tests (USP II, 37° C., 0.1N HCl). The samples tested are dry particles of APAP with a EUDRAGIT® L100-55/LUBRITAB® (40/60) coating (CR=25 wt %, blue x) and suspensions thereof stored for 6 months at 30° C. The suspensions have a continuous phase comprising either 60 wt % sorbitol (blue vertical line) or 60 wt % maltitol (orange circle) as the osmotic agent and adjusted to pH=3. The particles and suspensions are described in further detail in Example 8. The percent of drug released into the dissolution medium is graphed on the y-axis versus time (hours, x-axis). The in vitro dissolution profiles of the suspensions after storage do not substantially differ from the in vitro initial dissolution profile of the dry particles.

FIG. 24 graphically depicts the results of in vitro dissolution tests (USP II, 37° C., 0.1N HCl). The samples tested are dry particles of APAP with a EUDRAGIT® L100-55/EUDRAGIT® S100/LUBRITAB® (20/20/60) coating (CR=25 wt %, orange circle) and suspensions thereof stored for 6 months at 30° C. The suspensions have a continuous phase comprising either 60 wt % sorbitol (blue vertical line) or 60 wt % maltitol (blue x) as the osmotic agent and adjusted to pH=3. The particles and suspensions are described in further detail in Example 8. The percent of drug released into the dissolution medium is graphed on the y-axis versus time (hours, x-axis). The in vitro dissolution profiles of the suspensions after storage do not substantially differ from the in vitro initial dissolution profile of the dry particles.

FIG. 25A graphically depicts the results of in vitro dissolution tests (USP II, 37° C., 0.1N HCl). The samples tested are dry particles of APAP with a EUDRAGIT® L100/LUBRITAB® (40/60) coating (CR=25 wt %, purple x) and suspensions thereof stored for 6 months at 30° C. The suspensions have a continuous phase comprising either 60 wt % sorbitol (light blue squares) or 60 wt % maltitol (dark blue diamonds) as the osmotic agent and adjusted to pH=3. The particles and suspensions are described in further detail in Example 8. The percent of drug released into the dissolution medium is graphed on the y-axis versus time (hours, x-axis). The in vitro dissolution profiles of the suspensions after storage do not substantially differ from the in vitro initial dissolution profile of the dry particles. FIG. 25B graphically depicts the results of in vitro dissolution tests (USP II, 37° C., 0.1N HCl or 0.1N HCl for 3 hours then PB pH 6). The samples tested are suspensions of 60 wt % sorbitol (pH=3) stored for 3 months at 30° C. The APAP particles in the suspension have a EUDRAGIT® L100/LUBRITAB® (40/60) coating (CR=25 wt %). The in vitro dissolution tests occurred at 0.1N HCl for the duration of the test (blue diamonds) or at 0.1N HCl for 3 hours and then at pH 6 (red squares). The particles and suspensions are described in further detail in Example 8. The percent of drug released into the dissolution medium is graphed on the y-axis versus time (hours, x-axis).

FIG. 26 graphically depicts the results of in vitro dissolution tests (USP II, 37° C., 0.1N NCI). The samples tested are particles of APAP in suspension (sorbitol 60% adjusted to pH=3) after 1 h curing at 60° C. The particles have a EUDRAGIT® L100/LUBRITAB® (40/60) coating at a coating ratio of 15 wt % (blue diamond), 20 wt % (green triangle) or 25 wt % (red square). The particles are described in further detail in Example 8. The percent of drug released into the dissolution medium is graphed on the y-axis versus time (hours, x-axis).

FIG. 27 graphically depicts the results of in vitro dissolution tests (USP II, 37° C., 0.1N HCl). The percent of drug released into the dissolution medium is graphed on the y-axis versus time (hours, x-axis). The samples tested are dry particles of APAP with a EUDRAGIT® L100/LUBRITAB® (40/60) coating at a coating ratio of 25 wt % after various lengths of curing: uncured (light blue vertical lines), 0.5 hours (dark blue diamonds), 1 hour (red squares), 2 hours (green triangles), 4 hours (purple x), 6 hours (blue x), 24 hours (orange circles). The particles are described in further detail in Example 8

FIG. 28A, FIG. 28B, FIG. 28C, FIG. 28D, and FIG. 28E show the stability of APAP EUDRAGIT® L100/LUBRITAB® particles in suspension as a function of polymer/wax ratio. Further details are found in Example 9. FIG. 28A graphically depicts the results of in vitro dissolution tests (USP II, 37° C., 0.1N HCl). The samples tested are dry particles with a Eudragit L100/Lubritab (100/0) coating at a coating ratio of 25 wt % (blue vertical lines) and a suspension thereof stored for 1 week at 30° C. (red squares) or 1 month at 30° C. (orange circles). The percent of drug released into the dissolution medium is graphed on the y-axis versus time (hours, x-axis). The in vitro dissolution profile of the suspension after storage differs from the initial dissolution profile of the dry particles. FIG. 28B graphically depicts the results of in vitro dissolution tests (USP II, 37° C., 0.1N HCl). The samples tested are dry particles with a Eudragit L100/Lubritab (80/20) coating at a coating ratio of 25 wt % (blue diamonds) and suspensions thereof stored for 1 week at 30° C. (orange circles), 2 months at 30° C. (red squares), or 3 months at 30° C. (green triangles). The percent of drug released into the dissolution medium is graphed on the y-axis versus time (hours, x-axis). The in vitro dissolution profile of the suspension after storage differs from the initial dissolution profile of the dry particles. FIG. 28C graphically depicts the results of in vitro dissolution tests (USP II, 37° C., 0.1N HCl). The samples tested are dry particles with a Eudragit L100/Lubritab (60/40) coating at a coating ratio of 25 wt % (green triangles) and suspensions thereof stored for 1 month at 30° C. (red squares) or 2 months at 30° C. (blue stars). The percent of drug released into the dissolution medium is graphed on the y-axis versus time (hours, x-axis). The in vitro dissolution profile of the suspension after storage differs from the initial dissolution profile of the dry particles. FIG. 28D graphically depicts the results of in vitro dissolution tests (USP II, 37° C., 0.1N HCl). The samples tested are dry particles with a Eudragit L100/Lubritab (40/60) coating at a coating ratio of 25 wt % (red squares) and suspensions thereof stored for 6 months at 30° C. (blue stars). The percent of drug released into the dissolution medium is graphed on the y-axis versus time (hours, x-axis). The in vitro dissolution profiles of the suspensions after storage do not substantially differ from the in vitro initial dissolution profile of the dry particles. FIG. 28E graphically depicts the results of in vitro dissolution tests (USP II, 37° C., 0.1N HCl). The samples tested are dry particles with a Eudragit L100/Lubritab (20/80) coating at a coating ratio of 25 wt % (purple X) and suspensions thereof stored for 6 months at 30° C. (blue vertical line). The percent of drug released into the dissolution medium is graphed on the y-axis versus time (hours, x-axis). The in vitro dissolution profiles of the suspensions after storage do not substantially differ from the in vitro initial dissolution profile of the dry particles.

FIG. 29A and FIG. 29B show the stability of APAP and Metformin HCl particles respectively with L100-Lubritab (40/60) coating as a function of external osmolality. Further details are found in Example 9. FIG. 29A graphically depicts the results of in vitro dissolution tests (USP II, 37° C., 0.1N HCl). The samples tested are APAP particles with a Eudragit L100/Lubritab (40/60) coating at a coating ratio of 25 wt % as a suspension stored for 1 week (blue stars), 1 month (blue diamonds), or 6 months (purple X). The drug free fraction is graphed on the y-axis versus maltitol content (wt %, x-axis). FIG. 29B graphically depicts the results of in vitro dissolution tests (USP II, 37° C., 0.1N HCl). The samples tested are Metformin HCl particles with a Eudragit L100/Lubritab (40/60) coating at a coating ratio of 25 wt % as a suspension stored for 1 week (purple X), 2 weeks (blue diamonds), 1 month (red squares), 2 months (green triangles), 3 months (blue stars), or 6 months (orange circles). The drug free fraction is graphed on the y-axis versus maltitol content (wt %, x-axis).

FIG. 30 graphically depicts the results of in vitro dissolution tests (USP II, 37° C., 0.1N HCl). The samples tested are dry particles of Metformin HCl with a Eudragit L100/Lubritab (40/60) coating at a coating ratio of 25 wt % (green triangles) and suspensions thereof with 60 wt % sorbitol and adjusted to pH=3 stored for 1 week at 30° C. (blue diamonds), 1 month at 30° C. (red squares), or 6 months at 30° C. (orange circles). The percent of drug released into the dissolution medium is graphed on the y-axis versus time (hours, x-axis). The in vitro dissolution profiles of the suspensions after storage do not substantially differ from the in vitro initial dissolution profile of the dry particles. Further details are found in Example 9.

FIG. 31 graphically depicts the results of in vitro dissolution tests (USP II, 37° C., 0.1N HCl). The samples tested are dry particles of Guaifenesin with a Eudragit L100/Lubritab (40/60) coating at a coating ratio of 25 wt % (blue stars) and suspensions thereof with 60 wt % maltitol and adjusted to pH=3 stored for 1 month at 30° C. (purple X), 2 months at 30° C. (red line), or 6 months at 30° C. (purple diamonds). The percent of drug released into the dissolution medium is graphed on the y-axis versus time (hours, x-axis). The in vitro dissolution profiles of the suspensions after storage do not substantially differ from the in vitro initial dissolution profile of the dry particles. Further details are found in Example 9.

FIG. 32 graphically depicts the results of in vitro dissolution tests (USP II, 37° C., 0.1N HCl). The samples tested are dry particles of Chlorpheniramine.maleate with a Eudragit L100/Lubritab (40/60) coating at a coating ratio of 25 wt % (blue diamonds) and suspensions thereof with 60 wt % sorbitol and adjusted to pH=3 stored for 1 week at 30° C. (red squares), 2 months at 30° C. (blue stars), or 3 months at 30° C. (orange circles). The percent of drug released into the dissolution medium is graphed on the y-axis versus time (hours, x-axis). The in vitro dissolution profiles of the suspensions after storage do not substantially differ from the in vitro initial dissolution profile of the dry particles. Further details are found in Example 9.

FIG. 33 graphically depicts the results of in vitro dissolution tests (USP II, 37° C., 0.1N HCl). The samples tested are dry particles of Dexmethylphenidate HCl with 50% of the dose from IR-coated particles (CA/TEC 90/10) and 50% from DR particles (CAB/S100/L100/CO 60/10/15/15) (dark blue line) and suspensions thereof with 85 wt % Lycasin and adjusted to pH=3 stored for 1 week at 30° C. (purple line), 1 month at 30° C. (red line), or 6 months at 30° C. (light blue line). The percent of drug released into the dissolution medium is graphed on the y-axis versus time (hours, x-axis). The in vitro dissolution profiles of the suspensions after storage do not substantially differ from the in vitro initial dissolution profile of the dry particles. Further details are found in Example 10.

FIG. 34 graphically depicts the results of in vitro dissolution tests (USP II, 37° C., 0.1N HCl or Phosphate 50 mM pH 6.8). The samples tested are dry particles of APAP with a Eudragit L100/CAB (40/60) coating at a coating ratio of 30 wt % (purple X, pH6.8; pink line, 0.1N HCl) and suspensions thereof in water adjusted to pH=3 (orange circles) or with 60 wt % maltitol adjusted to pH=3 stored for 1 week at 30° C. (blue diamonds) or 1 month at 30° C. (red squares). The percent of drug released into the dissolution medium is graphed on the y-axis versus time (hours, x-axis). The in vitro dissolution profiles of the suspensions after storage do not substantially differ from the in vitro initial dissolution profile of the dry particles dissolved in 0.1N HCl. Further details are found in Example 10.

FIG. 35 graphically depicts the results of in vitro dissolution tests (USP II, 37° C., 0.1N HCl). The samples tested are dry particles of APAP with a Eudragit L100-55/CAB (40/60) coating at a coating ratio of 30 wt % (orange circles) and suspensions thereof with 60 wt % maltitol adjusted to pH=3 stored for 1 week at 30° C. (blue diamonds), or 1 month at 30° C. (green triangles). The percent of drug released into the dissolution medium is graphed on the y-axis versus time (hours, x-axis). The in vitro dissolution profiles of the suspensions after storage do not substantially differ from the in vitro initial dissolution profile of the dry particles. Further details are found in Example 10.

FIG. 36 graphically depicts the results of in vitro dissolution tests (USP II, 37° C., 0.1N HCl). The samples tested are dry particles of Metformin HCl with a Eudragit L100-55/EC std100 (40/60) coating at a coating ratio of 30 wt % (light blue line) and suspensions thereof with 60 wt % maltitol and adjusted to pH=3 stored for 1 month at 30° C. (dark blue line) or 6 months at 30° C. (green line). The percent of drug released into the dissolution medium is graphed on the y-axis versus time (hours, x-axis). The in vitro dissolution profiles of the suspensions after storage do not substantially differ from the in vitro initial dissolution profile of the dry particles. Further details are found in Example 10.

### DETAILED DESCRIPTION

The present disclosure provides drug-containing particles that have minimal loss of the drug from the particle when the particle is formulated as a suspension in a continuous phase that is not saturated by the drug. The present disclosure also provides compositions that are suspensions comprising a plurality of drug-containing particles dispersed in a continuous phase that is not saturated by the drug. An advantage of the compositions disclosed herein is that they are stable when stored for weeks or months, even at temperatures above 20-25° C. (i.e., room temperature). Other aspects of the particles and compositions of the present disclosure are described more thoroughly below.

Several definitions that apply throughout this disclosure will now be presented. As used herein, “about” refers to numeric values, including whole numbers, fractions, percentages, etc., whether or not explicitly indicated. The term “about” generally refers to a range of numerical values, for instance, ±0.5-1%, ±1-5% or ±5-10% of the recited value, that one would consider equivalent to the recited value, for example, having the same function or result.

The term “comprising” means “including, but not necessarily limited to”; it specifically indicates open-ended inclusion or membership in a so-described combination, group, series and the like. The terms “comprising” and “including” as used herein are inclusive and/or open-ended and do not exclude additional, unrecited elements or method processes. The term “consisting essentially of” is more limiting than “comprising” but not as restrictive as “consisting of.” Specifically, the term “consisting essentially of” limits membership to the specified materials or steps and those that do not materially affect the essential characteristics of the claimed invention. For example, a particle consisting essentially of a drug-containing core and a sustained-release coating could not include a second, unrecited coating unless that coating did not materially affect the drug-release profile imparted to the particle by the sustained-release coating.

A “coating” is a composition that is layered over something, for example, a drug-containing core. As used herein, the terms “coating” and “layer” may be used interchangeably. The amount of each component in a coating is expressed as a percent of the total weight of the coating. For example, an ethylcellulose/polyvinylpyrrolidone/castor oil (80 wt %/10 wt %/10 wt %) coating is a coating consisting of 80 weight percent (wt %) ethylcellulose, 10 wt % polyvinylpyrrolidone, and 10 wt % castor oil. If units are omitted (e.g., 80/10/10), it is understood that the amounts indicated are weight percent. Particles of the present disclosure may have one or more coatings. As such, a coating may or may not be adjacent to the particle's core and may or may not be on the outside of the particle (i.e., an outer coating).

The term “coating ratio” refers to the weight of a coating applied to a particle. Coating ratio is abbreviated as “CR.” The coating's weight is expressed as a percent of the total weight of the particle after the coating has been applied. For example, a coating ratio of 25 wt % indicates that a coating was applied to a particle (whether the particle was initially uncoated or previously coated) that accounts for 25% of the coated particle's weight.

Particles of the present disclosure have a drug-containing core. The term “drug-containing core” refers to the innermost portion of the particle that contains a drug. In some examples, a drug-containing core may comprise a crystalline form of a drug. In other examples, a drug-containing core may be a drug-coated pellet or bead, or a drug-ion exchange resin.

The terms “extended release,” “modified release,” and “sustained release” are interchangeable. Modified release drug delivery systems are designed to deliver drugs at a specific time or over a period of time after administration, or at a specific location in the body. The USP defines a modified release system as one in which the time course or location of drug release or both, are chosen to accomplish objectives of therapeutic effectiveness or convenience not fulfilled by conventional immediate release dosage forms. An extended release or sustained release product is formulated to make the drug available over an extended period after ingestion, thus allowing a reduction in dosing frequency compared to a drug presented as a conventional dosage form, e.g. a solution or an immediate release dosage form.

The term “gastro-soluble” polymer refers to a polymer that dissolves in a stomach of healthy human subject.

The term “osmolality ratio of a composition” as used herein means a ratio between the osmolality of an external phase (e.g., the continuous phase of a composition) and the osmolality of a saturated solution of the drug in water. Osmolality is typically measured at ambient temperature and expressed as number of osmoles or milliosmoles of any water-soluble compound per kg of a continuous phase.

A “suspension of particles,” as used herein, refers to a plurality of drug-containing particles dispersed in a continuous phase. The particles may or may not be homogenously dispersed in the continuous phase.

A “continuous phase” or “liquid phase” as used herein, refers to the fluid or liquid phase of the suspension in which the particles are dispersed. The continuous phase may not be saturated by the drug. “Not saturated by the drug” as used herein refers to the amount of drug in the continuous phase being less than the amount at which the continuous phase is saturated with the drug at the temperature at which the saturation is measured. The temperature at which saturation is measured corresponds to the temperature at which stability is performed. In an example, saturation is measured at 30° C.

A “stable particle,” as used herein, refers to a particle that can be dispersed in a continuous phase that is not saturated by the drug and has an osmolality greater than the osmolality of a saturated solution of the drug in water, and then stored as that suspension for a period of time without a significant amount of the drug from the particle appearing in the continuous phase of the suspension.

A “stable suspension,” as used herein, refers to a suspension of drug-containing particles, wherein the suspension can be stored for a period of time without a significant amount of drug from the particle appearing in the continuous phase of the suspension. For example, a stable suspension can be a suspension that initially contains no drug in the continuous phase the amount of drug in the continuous phase of the suspension is reduced by a factor of 2, preferably by a factor of 5 and more preferably by a factor of 10 compared to the saturation of the continuous phase by said API. Other measures of a stable suspension include, but are not limited to, a stable in vitro dissolution profile.

A “stable in vitro dissolution profile,” as used herein, refers to an in vitro dissolution profile that does not differ from the in vitro dissolution profile of an initial suspension, or the in vitro dissolution profile of the dry particles prior to forming a suspension, by ±15%. For example, a stable dissolution profile for sustained release formulations does not differ by more than +115% over the whole dissolution profile.

Abbreviations as used herein refer to the following: EC: EthylCellulose, PVP: PolyVinylPyrollidone, CO: Castor Oil, CAB: Cellulose Acetate Butyrate, CA: Cellulose Acetate, LR: Layering Ratio, CR: Coating Ratio, FF: Free Fraction, and HPLC: High Performance Liquid Chromatography.

I. Stable Particles with Gastro-Soluble Coatings and Stable Solutions Thereof

A. Particles with Gastro-Soluble Coating

The present disclosure provides a particle that may limit diffusion out of the particle with the particle is dispersed in a continuous phase and stored as a suspension for a period of time. For example, the particle may include a drug-containing core and an outer layer covering the core. The outer layer may include a hydrophobic compound and a gastro-soluble polymer.

The drug-loaded particles may be obtained by various techniques known in the art. In some embodiments, the particles may be obtained by techniques including, but not limited to, agglomeration in the molten state, such as the Glatt ProCell™ technique, extrusion and spheronization, wet granulation, compacting, granulation and spheronization, where the spheronization is carried out (for example) in a fluidized bed apparatus equipped with a rotor, in particular using the Glatt CPS™ technique, spraying (for example) in a fluidized bed type apparatus equipped with zig-zag filter, in particular using the Glatt MicroPx™ technique, or spraying (for example) in a fluidized bed apparatus optionally equipped with a partition tube or Wurster tube. In an example, the particles are obtained using a fluidized bed coater equipped with a Wurster insert to coat cellulose spheres with the drug and further coat the drug loaded core with a functional coating.

Each particle may include a drug-containing core. The drug-containing core may include a drug-coated pellet, a bead or a resin. In an embodiment, the drug-containing core may be a drug-coated pellet or bead. Non-limiting examples of the bead or pellet include, microcrystalline cellulose (such as Avicel™ from FMC Biopolymer, Cellet™ from Pharmatrans or Celphere™ from Asahi Kasei), calcium carbonate (such as Omyapure™ 35 from Omya), dicalcium phosphate (such as Dicafos™ AC 92-12 from Budenheim) or tricalcium phosphate (such as Tricafos™ SC93-15 from Budenheim); composite spheres or granules, spheres of calcium carbonate and starch (such as Destab™ 90 S Ultra 250 from Particle Dynamics) or spheres of calcium carbonate and maltodextrin (such as Hubercal™ CCG4100 from Huber); or combinations thereof. The core may also comprise other particles of pharmaceutically acceptable excipients such as particles of hydroxypropyl cellulose (such as Klucel™ from Aqualon Hercules), guar gum particles (such as Grinsted™ Guar from Danisco), xanthan particles (such as Xantural™ 180 from CP Kelco). According to specific embodiments, the core is a cellulose microsphere, such as Cellets™90, Cellets™127, Cellets™100, Cellets™ 175, Cellets™ 200, Cellets™ 263, Cellets™ 350 or Cellets™ 500 marketed by Pharmatrans, or also Celphere™ SCP 100, Celphere™ CP 102, Celphere™ CP 203, Celphere™ CP305, Celphere™ CP507 from Asahi Kasei Corporation.

The drug may be incorporated within or layered on the core to create the drug-containing core. The drug may be a small molecule drug. Non-limiting examples of the drug include Metformin HCl, acetaminophen (APAP), or guaifenesin. In some embodiments, the drug-containing core comprises a crystalline form of the drug.

The core may optionally include a binder. In some embodiments, the binder may be in an amount of less than about 5 wt % compared with the amount of drug in the core. In an embodiment, the binder may be in an amount of less than about 3 wt % compared with the amount of drug in the core. In an embodiment, the binder may be in an amount of less than about 2 wt % compared with the amount of drug in the core. In an embodiment, the binder may be in an amount of less than about 1 wt % compared with the amount of drug in the core. In an embodiment, the core may not include a binder.

Non-limiting examples of suitable binders include a cellulose derivative, povidone, maltodextrin, sodium alginate, gelatin, starch, a polyacrylamide, polyvinyloxoazolidone, a polyvinylalcohol, a C₁₂-C₁₈ fatty acid alcohol, polyethylene glycol, or a polyol. In an embodiment, the cellulose derivative may be hydroxypropylcellulose or hydroxypropylmethylcellulose.

The core may have a diameter in the range of about 50 μm to about 700 μm. In some embodiments, the diameter of the core may range from about 50 μm to about 100 μm, about 100 μm to about 300 μm, about 200 μm to about 400 μm, about 300 μm to about 500 μm, about 400 μm to about 600 μm, or about 500 μm to about 700 μm. In one embodiment, the core has a diameter of 100 μm to 500 μm.

Each of the particles further includes an outer layer covering the drug-containing core that includes a hydrophobic compound and a gastro-soluble polymer. The hydrophobic compound may be lipidic with a melting temperature (Tₘ) greater than or equal to about 50° C. In an embodiment, the hydrophobic compound has a Tₘ between about 50° C. and about 90° C. Non-limiting examples of the hydrophobic compound include hydrogenated vegetable oils, vegetable waxes, wax yellow, wax white, wax microcrystalline, lanolin, anhydrous milk fat, hard fat suppository base, lauroyl macrogolglycerides, cetyl alcohols, polyglyceryl diisostearate, diesters of glycerol with at least one fatty acid, or triesters of glycerol with at least one fatty acid. In an embodiment, the hydrophobic compound is LUBRITAB®.

The gastro-soluble polymer may include a minimum of 50% molar ratio of methyl methacrylate and a maximum of 50% molar ratio of an amino alkyl methacrylate. The gastro-soluble polymer may further exhibit a Tg>50° C. and an overall MW>50 000 g/mol. A non-limiting example of the gastro-soluble polymer includes a dimethyl amino ethyl methacrylate: methyl ethacrylate co-polymer. In an embodiment, the gastro-soluble polymer is KOLLICOAT® SmartSeal 30 D. When the gastro-soluble polymer is KOLLICOAT® SmartSeal 30 D, it is difficult to add a hydrophobic compound such as wax in the formulation, especially in such amounts that allows stability of the coating in liquid as in the particles. Therefore, the KOLLICOAT® SmartSeal may be solubilized in a hot solvent and then mixed with the hydrophobic compound.

The hydrophobic compound and gastro-soluble polymer may be present in a weight ratio of at least 2.3. In some embodiments, the hydrophobic compound and gastro-soluble polymer are present in a weight ratio of at least 3. In an embodiment, the hydrophobic compound and gastro-soluble polymer are present in a weight ratio of at least 4. In another embodiment, the hydrophobic compound and gastro-soluble polymer are present in a weight ratio of at least 5. In an embodiment, the hydrophobic compound and gastro-soluble polymer are present in a weight ratio of about 4. In some embodiments, the hydrophobic compound and gastro-soluble polymer are present in a weight ratio of at least 2.3 to 3, at least 2.3 to 4, at least 2.3 to 5, at least 3 to 4, at least 3 to 5, or at least 4 to 5. In some embodiments, the hydrophobic compound and gastro-soluble polymer are present in a weight ratio of about 2.3 to 3, about 2.3 to 4, about 2.3 to 5, about 3 to 4, about 3 to 5, or about 4 to 5.

The outer layer may include about 70 wt % or more of the hydrophobic compound and about 30 wt % or less of the gastro-soluble polymer. In an embodiment, the outer layer includes about 70 wt % to about 90 wt % of the hydrophobic compound and about 10 wt % to about 30 wt % or less of the gastro-soluble polymer. In another embodiment, the outer layer includes about 75 wt % to about 85 wt % of the hydrophobic compound and about 15 wt % to about 25 wt % or less of the gastro-soluble polymer.

The particle may further include one or more layers between the drug-containing core and the outer layer. In some embodiments, one or more of the layers is a sustained-release layer or a delayed release layer, for example, as seen in Examples 4 and 5.

The coating ratio of the outer layer is about 10 wt % to about 60 wt %. In an embodiment, the coating ratio of the outer layer is at least about 10 wt %. In an embodiment, the coating ratio of the outer layer is at least about 20 wt %. In an embodiment, the coating ratio of the outer layer is at least about 30 wt %. In an embodiment, the coating ratio of the outer layer is at least about 40 wt %. In an embodiment, the coating ratio of the outer layer is at least about 50 wt %. In an embodiment, the coating ratio of the outer layer is less than about 60 wt %. In one embodiment, the coating ratio is about 30 wt %.

In various embodiments, the suspension has an osmolality greater than a saturated solution of the drug in water and greater than 1300 mOsm/kg. In additional embodiments, the suspension has a pH greater than 7.0. For example, the suspension has an osmolality greater than 1300 mOsm/kg and a pH greater than 7.0. These conditions may allow the particles to be stable after being stored as a suspension for an extended period of time. For example, after storage of the particles as a suspension for at least one month at about 30° C., the suspension has a stable in vitro dissolution profile. The particles may be stable after being stored as a suspension for at least 3 months. The particles may be stable after being stored as a suspension for at least 6 months. The particles may be stable after being stored as a suspension for at least 1 year. The particles may be stable after being stored as a suspension for at least 2 years.

After storage of the particles as a suspension for at least one month at about 30° C., the amount of drug in the continuous phase of the suspension is reduced at least by a factor of 2 compared to the saturation of the continuous phase by the drug. In an embodiment, the amount of drug in the continuous phase of the suspension is reduced at least by a factor of 5 compared to the saturation of the continuous phase by the drug. In another embodiment, the amount of drug in the continuous phase of the suspension is reduced at least by a factor of 10 compared to the saturation of the continuous phase by the drug.

B. Suspension with Gastro-Soluble Coated Particles

The gastro-soluble coated particles are formulated and stored as a suspension. The suspension includes a plurality of the particles dispersed in a continuous phase. The continuous phase is not saturated by the drug contained in the particles, includes an osmotic agent, and has a pH that is above the gastro-soluble polymer's pKa.

In various embodiments, the continuous phase is not saturated by the drug. In an embodiment, the suspension includes less than about 10 wt % particles. In an example, the suspension includes about 10 wt % particles. In another example, the suspension includes about 5 wt % of particles.

The amount of drug in the continuous phase of the suspension may be a factor of 2 less than the saturation of the continuous phase by the drug. In an embodiment, the amount of drug in the continuous phase of the suspension may be a factor of 5 less than the saturation of the continuous phase by the drug. In another embodiment, the amount of drug in the continuous phase of the suspension may be a factor of 10 less than the saturation of the continuous phase by the drug.

Without being limited to a particular theory, a high osmolality in the continuous phase aids in maintaining the stability of the solution and limiting the release of the drug from the particle to continuous phase during storage. In an embodiment, the osmolality of the continuous phase may be greater than a saturated solution of the drug in water. In another embodiment, the osmolality of the continuous phase may be greater than about 1300 mOsm/kg. In yet another embodiment, the osmolality of the continuous phase may be higher than a saturated solution of the drug in water and higher than a minimum value of 1300 mOsm/kg.

The continuous phase has a pH that is above the gastro-soluble polymer's pKa. In an embodiment, the continuous phase has a pH above 5.0. In an embodiment, the continuous phase has a pH above 7.0. In another embodiment, the continuous phase has a pH greater than 7.5.

In various embodiments, the continuous phase includes at least one osmotic agent. The osmotic agent may include a polyol, a sugar, a salt or mixtures thereof. Non-limiting examples of the osmotic agent is a sugar alcohol, citrate, polydextrose, fructose, glucose, maltose, or sucrose. The sugar alcohol may be maltitol, sorbitol, erythritol, sorbitol, xylitol, isomalt, or mannitol. The osmotic agent may be present in the continuous phase in an amount of at least 30 wt %. In an embodiment, the osmotic agent may be present in the continuous phase in an amount of at least 40 wt %. In an embodiment, the osmotic agent may be present in the continuous phase in an amount of at least 50 wt %. In an embodiment, the osmotic agent may be present in the continuous phase in an amount of at least 60 wt %. In one embodiment, the continuous phase may include about 60 wt % maltitol.

In some embodiments, the continuous phase further includes a suspending agent. The suspending agent may be any suspending agent commonly used in liquid pharmaceutical formulations and that can be found in the “Handbook of Pharmaceutical Excipients” 8th edition. Non-limiting examples of suspending agents include cellulose derivatives such as co-processed spray dried forms of microcrystalline cellulose and carboxymethyl cellulose sodium, hydroxypropyl cellulose, hydroxyethyl cellulose, hydroxypropylmethyl cellulose, methylcellulose, carboxymethyl cellulose and its salts/derivatives, and microcrystalline cellulose; carbomers; gums such as locust bean gum, xanthan gum, tragacanth gum, arabinogalactan gum, agar gum, gellan gum, guar gum, apricot gum, karaya gum, sterculia gum, acacia gum, gum arabic, and carrageenan; pectin; dextran; gelatin; polyethylene glycols; polyvinyl compounds such as polyvinyl acetate, polyvinyl alcohol, and polyvinyl pyrrolidone; sugar alcohols such as xylitol and mannitol; colloidal silica; or mixtures thereof. Co-processed spray dried forms of microcrystalline cellulose and carboxymethyl cellulose sodium have been marketed under the trade names Avicel® RC-501, Avicel® RC-581, Avicel® RC-591, and Avicel® CL-611. In an embodiment, the suspending agent may be xanthan gum. For example, the amount of xanthan gum may be about 0.1 wt % to about 1 wt %, about 0.1 wt % to about 0.5 wt %, or about 0.1 wt % to about 0.2 wt %.

The suspension may have a stable in vitro dissolution profile. The suspension may be stable after storage of the suspension for at least one month at about 30° C. For example, after storage of the composition in suspension for at least one month at about 30° C., the composition has a stable in vitro dissolution profile. The suspension may be stable after being stored for at least 3 months at about 30° C. The suspension may be stable after being stored for at least 6 months at about 30° C. The suspension may be stable after being stored for at least 1 year at about 30° C. The suspension may be stable after being stored for at least 2 years at about 30° C.

The amount of drug in the continuous phase of the suspension may be reduced at least by a factor of 2 compared to the saturation of the continuous phase by the drug. In an example, the amount of drug in the continuous phase of the suspension may be reduced at least by a factor of 5 compared to the saturation of the continuous phase by the drug. In another example, the amount of drug in the continuous phase of the suspension may be reduced at least by a factor of 10 compared to the saturation of the continuous phase by the drug.

II. Stable Solutions with Extended Release Particles

A. Extended Release Particles

The present disclosure further provides an extended release particle that when dispersed in a continuous phase and stored as a suspension is stable for an extended period of time. Each particle may include a drug-containing core and an outer layer covering the core. In an embodiment, each particle consists essentially of the drug-containing core and the outer layer covering the core. In an example, the particles are obtained using a fluidized bed coater equipped with a Wurster insert to coat cellulose spheres with the drug and further coat the drug loaded core with a functional coating.

The drug-containing core may include a drug-coated pellet, a bead or a resin. In an embodiment, the drug-containing core may be a drug-coated pellet or bead. Non-limiting examples of the bead or pellet include, microcrystalline cellulose (such as Avicel™ from FMC Biopolymer, Cellet™ from Pharmatrans or Celphere™ from Asahi Kasei), calcium carbonate (such as Omyapure™ 35 from Omya), dicalcium phosphate (such as Dicafos™ AC 92-12 from Budenheim) or tricalcium phosphate (such as Tricafos™ SC93-15 from Budenheim); composite spheres or granules, spheres of calcium carbonate and starch (such as Destab™ 90 S Ultra 250 from Particle Dynamics) or spheres of calcium carbonate and maltodextrin (such as Hubercal™ CCG4100 from Huber); or combinations thereof. The core may also comprise other particles of pharmaceutically acceptable excipients such as particles of hydroxypropyl cellulose (such as Klucel™ from Aqualon Hercules), guar gum particles (such as Grinsted™ Guar from Danisco), xanthan particles (such as Xantural™ 180 from CP Kelco). According to specific embodiments, the core is a cellulose microsphere, such as Cellets™90, Cellets™127, Cellets™100, Cellets™ 175, Cellets™ 200, Cellets™ 263, Cellets™ 350 or Cellets™ 500 marketed by Pharmatrans, or also Celphere™ SCP 100, Celphere™ CP 102, Celphere™ CP 203, Celphere™ CP305, Celphere™ CP507 from Asahi Kasei Corporation.

The drug may be incorporated within or layered on the core to create the drug-containing core. The drug-containing core may include a crystalline form of the drug. The drug may have a film/water partitioning coefficient that is less than 1 (i.e. a decimal logarithm less than 0). In an embodiment, the drug has a partitioning coefficient that is less than −0.5. The drug may be a small molecule drug. Non-limiting examples of the drug include Metformin HCl, pyridoxine HCl, phenylephrine HCl, losartan potassium, pseudoephedrine HCl, cetirizine di HCl, chlorpheniramine maleate, diphenhydramine HCl, fexofenadine HCl, sodium p-hydroxybenzoate HBr, sodium salicylate sodium valproate, caffeine, and combinations thereof.

The core may optionally include a binder. In some embodiments, the binder may be in an amount of less than about 5 wt % compared with the amount of drug in the core. In an embodiment, the binder may be in an amount of less than about 3 wt % compared with the amount of drug in the core. In an embodiment, the binder may be in an amount of less than about 2 wt % compared with the amount of drug in the core. In an embodiment, the binder may be in an amount of less than about 1 wt % compared with the amount of drug in the core. In an embodiment, the core may not include a binder.

Non-limiting examples of suitable binders include a cellulose derivative, povidone, maltodextrin, sodium alginate, gelatin, starch, a polyacrylamide, polyvinyloxoazolidone, a polyvinylalcohol, a C₁₂-C₁₈ fatty acid alcohol, polyethylene glycol, or a polyol. In an embodiment, the cellulose derivative may be hydroxypropylcellulose or hydroxypropylmethylcellulose.

The core may have a diameter in the range of about 50 μm to about 500 μm. In some embodiments, the diameter of the core may range from about 50 μm to about 100 μm, about 100 μm to about 300 μm, about 200 μm to about 400 μm, or about 300 μm to about 500 μm, about 400 μm to about 600 μm, or about 500 μm to about 700 μm. In one embodiment, the core has a diameter of 100 μm to 500 μm.

The extended release particles further include an outer layer covering the drug-containing core. The outer layer includes (i) up to 20 wt % of one or more water-soluble polymer and (ii) at least about 80 wt % of a mixture of at least one cellulosic derivative insoluble in the gastrointestinal tract and a plasticizer. In an embodiment, the outer layer includes up to 5 wt % of one or more water soluble polymers. In an embodiment, the outer layer includes up to 10 wt % of one or more water soluble polymers. In an embodiment, the outer layer includes up to 15 wt % of one or more water soluble polymers. In an embodiment, the outer layer includes up to 20 wt % of one or more water soluble polymers. In an embodiment, the outer layer includes at least about 80 wt % of a mixture of at least one cellulosic derivative insoluble in the gastrointestinal tract and a plasticizer. In an embodiment, the outer layer includes at least about 85 wt % of a mixture of at least one cellulosic derivative insoluble in the gastrointestinal tract and a plasticizer. In an embodiment, the outer layer includes at least about 90 wt % of a mixture of at least one cellulosic derivative insoluble in the gastrointestinal tract and a plasticizer. In an embodiment, the outer layer includes at least about 95 wt % of a mixture of at least one cellulosic derivative insoluble in the gastrointestinal tract and a plasticizer.

The water-soluble polymer may include, but is not limited to polyvinylpyrrolidone, a water-soluble cellulose derivative, a copolymer of N-vinyl-2-pyrrolidone and vinyl acetate, polyvinyl alcohol-polyethylene glycol graft copolymer, a polyacrylamide, a poly-N-vinylamide, a poly-N-vinyllactam, or a polyoxyethylene. In an embodiment, the water-soluble polymer may be polyvinylpyrrolidone or copovidone.

In some examples, the outer layer may include a polymer insoluble in the gastrointestinal tract, including but not limited to a cellulosic derivative, an acrylic derivative, and polyvinyl acetate. Non-limiting examples of cellulosic derivatives insoluble in the gastrointestinal tract include water-insoluble cellulose derivatives, such as ethyl cellulose or cellulose acetate butyrate.

The cellulosic derivative may be mixed with a plasticizer. Examples of suitable plasticizers include, without limit, castor oil, cutin, glycerol, a glycerol ester, a phthalate, a citrate, a sebacate, a cetyl alcohol ester, a malonate, triacetin, a butyrate, a succinate, a malate, a fumarate, a benzoate, an azelate, or an adipate. In an embodiment, the glycerol ester is an acetylated glyceride, glycerol monostearate, glyceryl triacetate, or glycerol tributyrate. In another embodiment, the phthalate is dibutyl phthalate, diethyl phthalate, dimethyl phthalate, or dioctyl phthalate. In an embodiment, the citrate is acetyltributyl citrate, acetyltriethyl citrate, tributyl citrate, or triethyl citrate. In another embodiment, the sebacate is diethyl sebacate or dibutyl sebacate. In additional embodiments, the malonate is diethyl malonate. In further embodiments, the succinate is dibutyl succinate. In an embodiment, the oxalate is diethyl oxalate. In another embodiment, the fumarate is diethyl fumarate. In one embodiment, at least one plasticizer is castor oil.

In an embodiment, the outer layer may include ethyl cellulose, polyvinylpyrrolidone, and castor oil. In another embodiment, the outer layer may include ethyl cellulose, copovidone, and castor oil. In yet another embodiment, the outer layer may include cellulose acetate butyrate, polyvinylpyrrolidone, and castor oil.

The coating ratio of the outer layer may be about 10 wt % or greater. In an embodiment, the coating ratio is about 15 wt % to about 45 wt %. In another embodiment, the coating ratio is about 15 wt % to about 30 wt %. In yet another embodiment, the coating ratio is about 30 wt % to about 45 wt %.

B. Suspension with Extended Release Particles

The extended release particles are formulated and stored as a suspension. The suspension includes a plurality of the particles dispersed in a continuous phase. The continuous phase is not saturated by the drug contained in the particles and includes at least one osmotic agent.

In various embodiments, the continuous phase is not saturated by the drug. In an embodiment, the suspension includes less than about 10 wt % particles. In an example, the suspension includes about 10 wt % particles. In another example, the suspension includes about 5 wt % of particles.

Without being limited to a particular theory, a high osmolality in the continuous phase aids in maintaining the stability of the solution and limiting the release of the drug from the particle to continuous phase during storage. In an embodiment, the osmolality of the continuous phase is higher than a saturated solution of the drug in water.

The continuous phase further includes at least one osmotic agent. The osmotic agent may include a polyol, a sugar, a salt or mixtures thereof. Examples of the osmotic agent include, without limit, a sugar, a sugar alcohol, a citrate, polydextrose, or combinations thereof. In an embodiment, the sugar may be fructose, glucose, maltose, or sucrose. In an embodiment, the sugar alcohol may be maltitol, sorbitol, erythritol, sorbitol, xylitol, mannitol, or isomalt. The osmotic agent may be present in the continuous phase in an amount of at least 30 wt %. In an embodiment, the osmotic agent may be present in the continuous phase in an amount of at least 40 wt %. In an embodiment, the osmotic agent may be present in the continuous phase in an amount of at least 50 wt %. In an embodiment, the osmotic agent may be present in the continuous phase in an amount of at least 60 wt %. In one embodiment, the continuous phase includes about 60 wt % maltitol. In another embodiment the continuous phase includes about 60 wt % sorbitol.

The suspending agent may be any suspending agent commonly used in liquid pharmaceutical formulations and that can be found in the “Handbook of pharmaceutical excipients” 8th edition. Non-limiting examples of suspending agents include cellulose derivatives such as co-processed spray dried forms of microcrystalline cellulose and carboxymethyl cellulose sodium, hydroxypropyl cellulose, hydroxyethyl cellulose, hydroxypropylmethyl cellulose, methylcellulose, carboxymethyl cellulose and its salts/derivatives, and microcrystalline cellulose; carbomers; gums such as locust bean gum, xanthan gum, tragacanth gum, arabinogalactan gum, agar gum, gellan gum, guar gum, apricot gum, karaya gum, sterculia gum, acacia gum, gum arabic, and carrageenan; pectin; dextran; gelatin; polyethylene glycols; polyvinyl compounds such as polyvinyl acetate, polyvinyl alcohol, and polyvinyl pyrrolidone; sugar alcohols such as xylitol and mannitol; colloidal silica; or mixtures thereof. Co-processed spray dried forms of microcrystalline cellulose and carboxymethyl cellulose sodium have been marketed under the trade names Avicel® RC-501, Avicel® RC-581, Avicel® RC-591, and Avicel® CL-611. In an embodiment, the suspending agent may be xanthan gum. For example, the amount of xanthan gum may be about 0.1 wt % to about 1 wt %, about 0.1 wt % to about 0.5 wt %, or about 0.1 wt % to about 0.2 wt %.

The suspension may be stable after storage of the suspension for at least one month at about 30° C. For example, after storage of the composition in suspension for at least one month at about 30° C., the composition has a stable in vitro dissolution profile. The suspension may be stable after being stored for at least 3 months at about 30° C. The suspension may be stable after being stored for at least 6 months at about 30° C. The suspension may be stable after being stored for at least 1 year at about 30° C. The suspension may be stable after being stored for at least 2 years at about 30° C.

In some embodiments, after storage of the particle for at least one month at about 40° C. and 65% relative humidity, as a suspension with an osmolality ratio of greater than 1, the suspension may have a stable in vitro dissolution profile

III. Stable Solutions with Delayed Release Particles

A. Delayed Release Particle

The present disclosure further provides a delayed release particle that when dispersed in a continuous phase and stored as a suspension is stable for an extended period of time. Each particle may include a drug-containing core and an outer layer covering the core. In an embodiment, each particle consists essentially of the drug-containing core and the outer layer covering the core. In an example, the particles are obtained using a fluidized bed coater equipped with a Wurster insert to coat cellulose spheres with the drug and further coat the drug loaded core with a functional coating.

The drug-containing core may include a drug-coated pellet, a bead or a resin. In an embodiment, the drug-containing core may be a drug-coated pellet or bead. Non-limiting examples of the bead or pellet include, microcrystalline cellulose (such as Avicel™ from FMC Biopolymer, Cellet™ from Pharmatrans or Celphere™ from Asahi Kasei), calcium carbonate (such as Omyapure™ 35 from Omya), dicalcium phosphate (such as Dicafos™ AC 92-12 from Budenheim) or tricalcium phosphate (such as Tricafos™ SC93-15 from Budenheim); composite spheres or granules, for example, spheres of calcium carbonate and starch (such as Destab™ 90 S Ultra 250 from Particle Dynamics) or spheres of calcium carbonate and maltodextrin (such as Hubercal™ CCG4100 from Huber); or combinations thereof. The core may also comprise other particles of pharmaceutically acceptable excipients such as particles of hydroxypropyl cellulose (such as Klucel™ from Aqualon Hercules), guar gum particles (such as Grinsted™ Guar from Danisco), xanthan particles (such as Xantural™ 180 from CP Kelco). According to specific embodiments, the core is a cellulose microsphere, such as Cellets™90, Cellets™127, Cellets™100, Cellets™ 175, Cellets™ 200, Cellets™ 263, Cellets™ 350 or Cellets™ 500 marketed by Pharmatrans, or also Celphere™ SCP 100, Celphere™ CP 102, Celphere™ CP 203, Celphere™ CP305, Celphere™ CP507 from Asahi Kasei Corporation.

The drug may be incorporated within or layered on the core to create the drug-containing core. The drug may be a small molecule drug. The drug-containing core may include a crystalline form of the drug. In various embodiments, the drug-containing core may include drugs that are ionized, non-ionized water-soluble, or weakly dosed.

The core may optionally include a binder. In some embodiments, the binder may be in an amount of less than about 5 wt % compared with the amount of drug in the core. In an embodiment, the binder may be in an amount of less than about 3 wt % compared with the amount of drug in the core. In an embodiment, the binder may be in an amount of less than about 2 wt % compared with the amount of drug in the core. In an embodiment, the binder may be in an amount of less than about 1 wt % compared with the amount of drug in the core. In an embodiment, the core may not include a binder.

Non-limiting examples of suitable binders include a cellulose derivative, povidone, maltodextrin, sodium alginate, gelatin, starch, a polyacrylamide, polyvinyloxoazolidone, a polyvinylalcohol, a C₁₂-C₁₈ fatty acid alcohol, polyethylene glycol, or a polyol. In an embodiment, the cellulose derivative may be hydroxypropylcellulose or hydroxypropylmethylcellulose.

The core may have a diameter in the range of about 50 μm to about 500 μm. In some embodiments, the diameter of the core may range from about 50 μm to about 100 μm, about 100 μm to about 300 μm, about 200 μm to about 400 μm, or about 300 μm to about 500 μm. In one embodiment, the core has a diameter of 100 μm to 300 μm, about 400 μm to about 600 μm, or about 500 μm to about 700 μm. In one embodiment, the core has a diameter of 100 μm to 500 μm.

An outer layer covers the drug-containing core of the particle. The outer layer includes one or more hydrophobic compounds that are crystalline in the solid state and one or more polymers carrying groups that are ionized at neutral pH.

The hydrophobic compound may have a Tₘ greater than or equal to about 40° C. The hydrophobic compound may have a Tₘ greater than or equal to about 50° C. Non-limiting examples of hydrophobic compounds include hydrogenated vegetable oils, vegetable waxes, wax yellow, wax white, wax microcrystalline, lanolin, anhydrous milk fat, hard fat suppository base, lauroyl macrogolglycerides, cetyl alcohols, polyglyceryl diisostearate, monoesters of glycerol with at least one fatty acid, diesters of glycerol with at least one fatty acid, or triesters of glycerol with at least one fatty acid. In one embodiment, the hydrophobic compound is LUBRITAB®.

In an embodiment, one or more of the hydrophobic compounds may be vegetable waxes, taken on their own or in mixtures with one another, such as those marketed under the marks DYNASAN® P60 and DYNASAN® 116, inter alia. In another embodiment, one or more of the hydrophobic compounds may be hydrogenated vegetable oils, taken on their own or in a mixture with one another. For example, the hydrogenated vegetable oils may include cottonseed oil, hydrogenated soybean oil, hydrogenated palm oil, and mixtures thereof. In other embodiments, one or more of the hydrophobic compounds may be monoesters and/or diesters and/or triesters of glycerol with at least one fatty acid, preferably behenic acid, taken by themselves or in a mixture with one another; and mixtures thereof.

The hydrophobic compound may include the products with the following tradenames (trademarks): Dynasan (Hydrogenated palm oil), Cutina (Hydrogenated castor oil), Hydrobase (Hydrogenated soybean oil), Dub (Hydrogenated soybean oil), Castorwax (Hydrogenated castor oil), Croduret (Hydrogenated castor oil), Carbowax, Compritol (Glyceryl behenate), Sterotex (Hydrogenated cottonseed oil), Lubritab (Hydrogenated cottonseed oil), Apifil (Wax yellow), Akofine (Hydrogenated cottonseed oil), Softtisan (Hydrogenated palm oil), Hydrocote (Hydrogenated soybean oil), Livopol (Hydrogenated soybean oil), Super Hartolan (Lanolin), MGLA (Anhydrous milk fat), Corona (Lanolin), Protalan (Lanolin), Akosoft (Suppository bases, Hard fat), Akosol (Suppository bases, Hard fat), Cremao (Suppository bases, Hard fat), Massupol (Suppository bases, Hard fat), Novata (Suppository bases, Hard fat), Suppocire (Suppository bases, Hard fat), Wecobee (Suppository bases, Hard fat), Witepsol (Suppository bases, Hard fat), Coronet, Lanol, Lanolin, Incromega (Omega 3), Estaram (Suppository bases, Hard fat), Estol, Suppoweiss (Suppository bases, Hard fat), Gelucire (Macrogolglycerides Lauriques), Precirol (Glyceryl Palm itostearate), Emulcire (Cetyl alcohol), Plurol diisostearique (Polyglyceryl Diisostearate), Geleol (Glyceryl Stearate), Hydrine et Monthyle; as well the additives which codes are the followings: E 901, E 907, E 903 and mixtures thereof; and mixtures thereof. In practice, the hydrophobic compound can be selected the products which tradenames (trademarks) are the followings: Dynasan P60, Dynasan 116, Dynasan 118, Cutina HR, Hydrobase 66-68, Dub, Compritol 888, Sterotex NF, Lubritab, and mixtures thereof.

The at least one polymer carrying groups that are ionized at neutral pH may be anionic enteric (co)polymers soluble in aqueous media at pH values above those encountered in the stomach. The polymer carrying groups that are ionized at neutral pH may be a methacrylic acid/alkyl (meth)acrylate copolymer. For example, the polymer may be EUDRAGIT® L100, EUDRAGIT® S100, EUDRAGIT® L100-55, or any combination thereof.

The weight ratio of the hydrophobic compound to the polymer carrying groups that are ionized at neutral pH may be greater than 1.5. In an embodiment, the weight ratio of the hydrophobic compound(s) to the polymer(s) carrying groups that are ionized at neutral pH is greater than 2.

In various embodiments, the outer layer comprises about 60 wt % to about 90 wt % of the hydrophobic compound(s) and about 10 wt % to about 40 wt % or less of the polymer(s) carrying groups that are ionized at neutral pH. In another embodiment, the outer layer comprises about 75 wt % to about 85 wt % of the hydrophobic compound and about 15 wt % to about 25 wt % or less of the polymer(s) carrying groups that are ionized at neutral pH. In some embodiments, the outer layer consists of about 70 wt % or more of the hydrophobic compound and about 30 wt % or less of the polymer(s) carrying groups that are ionized at neutral pH. In other embodiments, the outer layer consists of about 70 wt % to about 90 wt % of the hydrophobic compound and about 10 wt % to about 30 wt % or less of the polymer(s) carrying groups that are ionized at neutral pH.

The coating ratio of the outer layer to the particle may be about 15 wt % to about 45 wt %. In an embodiment, the coating ratio may be about 15 wt % to about 30 wt %. In another embodiment, the coating ratio may be about 30 wt % to about 45 wt

B. Suspension with Delayed Release Particles

The delayed release particles are formulated and stored as a suspension. The suspension includes a plurality of the particles dispersed in a continuous phase. The continuous phase is not saturated by the drug contained in the particles, includes at least one osmotic agent, and has a pH below 3.5.

In various embodiments, the continuous phase is not saturated by the drug. In an embodiment, the suspension includes less than about 10 wt % particles. In an example, the suspension includes about 10 wt % particles. In another example, the suspension includes about 5 wt % of particles.

Without being limited to a particular theory, a high osmolality in the continuous phase aids in maintaining the stability of the solution and limiting the release of the drug from the particle to continuous phase during storage. In an embodiment, the osmolality of the continuous phase may be greater than a saturated solution of the drug in water. In another embodiment, the osmolality of the continuous phase may be greater than about 1300 mOsm/kg. In yet another embodiment, the osmolality of the continuous phase may be higher than a saturated solution of the drug in water and higher than a minimum value of 1300 mOsm/kg.

In various embodiments, the continuous phase includes at least one osmotic agent. The osmotic agent may include a polyol, a sugar, a salt or mixtures thereof. Non-limiting examples of the osmotic agent are a sugar alcohol, citrate, polydextrose, fructose, glucose, maltose, sucrose, or combinations thereof. The sugar alcohol may be maltitol, sorbitol, erythritol, sorbitol, xylitol, isomalt, or mannitol. The osmotic agent may be present in the continuous phase in an amount of at least 30 wt %. In an embodiment, the osmotic agent may be present in the continuous phase in an amount of at least 40 wt %. In an embodiment, the osmotic agent may be present in the continuous phase in an amount of at least 50 wt %. In an embodiment, the osmotic agent may be present in the continuous phase in an amount of at least 60 wt %. In one embodiment, the continuous phase may include about 60 wt % maltitol.

In some embodiments, the continuous phase further includes a suspending agent. The suspending agent may be any suspending agent commonly used in liquid pharmaceutical formulations and that can be found in the “Handbook of pharmaceutical excipients” 8th edition. Non-limiting examples of suspending agents include cellulose derivatives such as co-processed spray dried forms of microcrystalline cellulose and carboxymethyl cellulose sodium, hydroxypropyl cellulose, hydroxyethyl cellulose, hydroxypropylmethyl cellulose, methylcellulose, carboxymethyl cellulose and its salts/derivatives, and microcrystalline cellulose; carbomers; gums such as locust bean gum, xanthan gum, tragacanth gum, arabinogalactan gum, agar gum, gellan gum, guar gum, apricot gum, karaya gum, sterculia gum, acacia gum, gum arabic, and carrageenan; pectin; dextran; gelatin; polyethylene glycols; polyvinyl compounds such as polyvinyl acetate, polyvinyl alcohol, and polyvinyl pyrrolidone; sugar alcohols such as xylitol and mannitol; colloidal silica; or mixtures thereof. Co-processed spray dried forms of microcrystalline cellulose and carboxymethyl cellulose sodium have been marketed under the trade names Avicel® RC-501, Avicel® RC-581, Avicel® RC-591, and Avicel® CL-611. In an embodiment, the suspending agent may be xanthan gum. For example, the amount of xanthan gum may be about 0.1 wt % to about 1 wt %, about 0.1 wt % to about 0.5 wt %, or about 0.1 wt % to about 0.2 wt %.

In some embodiments, the suspension with delayed release particles provides a pH dependent delayed release profile, even after storage for at least 1 month at 30° C.

The suspension may have a stable in vitro dissolution profile. For example, the suspension may be stable after storage for at least one month at about 30° C. A dissolution profile of the release of the stable suspension (during 1 month storage at 30° C., suspension at pH3) may be similar to the dissolution profile of release of the particles only. For example, after storage of the suspension for at least one month at about 30° C., the composition has a stable in vitro dissolution profile. The suspension may be stable after being stored for at least 3 months at about 30° C. The suspension may be stable after being stored for at least 6 months at about 30° C. The suspension may be stable after being stored for at least 1 year at about 30° C. The suspension may be stable after being stored for at least 2 years at about 30° C.

### EXAMPLES

The following examples are included to demonstrate preferred embodiments of the disclosure. It should be appreciated by those of skill in the art that the techniques disclosed in the examples that follow represent techniques discovered by the inventors to function well in the practice of the invention. Those of skill in the art should, however, in light of the present disclosure, appreciate that changes may be made in the specific embodiments that are disclosed and still obtain a like or similar result without departing from the spirit and scope of the disclosure. Therefore, all matter set forth or shown in the accompanying drawings is to be interpreted as illustrative and not in a limiting sense.

### Example 1

An immediate release composition was produced that is stable after storage for at least 6 months at 30° C. The composition is a suspension comprising a plurality of APAP-containing particles dispersed in a continuous phase. The particles have an APAP-containing core and an outer layer covering the core.

To manufacture the APAP-containing cores, 1260 g of APAP and 140 g of hydroxypropylcelullose (Klucel EF) were dissolved in a solvent mixture of water (1487.5 g) and ethanol (1487.5 g). Drug was layered onto 350 g of Cellet 127 microcrystalline cellulose cores in a Glatt GPC1.1 fluid bed coater using the Wurster process to produce a drug layer of 80% w/w. The layering conditions were controlled at a product temperature of 40° C., air flow of 50 m³/h, nozzle pressure of 2.8 bars and mean spray rate of 25 g/min.

An outer layer of a KOLLICOAT® Smartseal 30 D/LUBRITAB® (20 wt %/80 wt %) coating was then applied to the APAP cores to produce the final particle. 171.4 g of KOLLICOAT® Smartseal 30 D (solid content 30% w/w) and 120 g of hydrogenated cottonseed oil (LUBRITAB®) was dissolved in isopropanol (1422.9 g) heated at 78° C. The film was coated onto 400 g of previously prepared APAP cores in a Glatt GPC1.1 fluid bed coater using the Wurster process to produce a coating ratio of 30 wt %. The coating conditions were controlled at product temperature of 40° C., air flow of 65 m³/h, nozzle pressure of 1.6 bars and mean spray rate of 10 g/min.

Prior to preparing suspensions of APAP final particles, the continuous phase was prepared by manufacturing stock solutions of maltitol, xanthan gum, and phosphate buffer. 350.49 g of maltitol were dissolved in 150.5 g of water heated for about 1 hour at 60° C. In a separate container, 2.039 g of xanthan gum was dissolved in 98.017 g of water under moderate stirring until completely dissolved. A 1.18M phosphate buffer was prepared by solubilizing 8 g of KH₂PO₄ in 42.06 g of water and by incorporating 11.90 g of NaOH. This resulted in a stock solution adjusted at pH 7.6. The continuous phase was prepared in a 1 L beak by mixing 385.96 g of maltitol stock solution with 44.18 g of xanthan gum stock solution, 19.13 g of phosphate buffer and 0.84 g of water. The blend was homogenized under gentle stirring for 10-15 minutes at room temperature. The final suspension was prepared by mixing 90 g of the continuous phase with 10 g of the APAP final particles. Homogenization was performed under moderate stirring for 5 minutes prior to transfer into a PET bottle. The pH of the suspension was measured to be about pH 7.2. Compositions were stored at 30° C. for further evaluation.

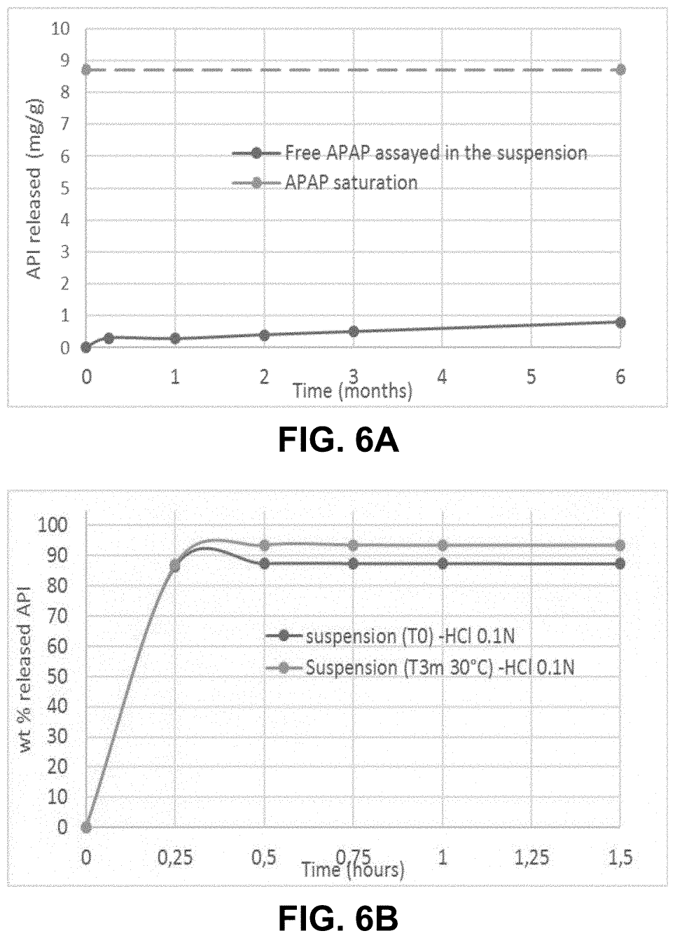

At different time points, the stability of the composition was evaluated by measuring APAP solubilization into the continuous phase and/or the in vitro dissolution profile of the composition, and comparing the values to the solution prior to storage and/or to dry particles. Briefly, a sample volume of the composition was withdrawn from the container being stored at 30° C. after manual shaking of the bottle. Dissolution profiles were obtained by introducing a sample volume into a USP Type II apparatus equipped with 1 L vessels filled with 900 mL 0.1N HCl. The vessels were maintained at 37° C. and the rotational speed of the paddles set at 100 RPM. The solubilized APAP fraction in the continuous phase of the composition was assayed by HPLC after filtering about 3 ml of composition onto a 10 μm filter. As shown in FIG. 6, the composition has stable behavior in suspension for at least 6 months of storage at 30° C. with a solubilized content of APAP in the continuous phase of the suspension reduced by a factor of 10 compared to the saturation of the continuous phase by the drug. Bioavailability of the drug is not affected by the coating as the immediate release profile is obtained and maintained overtime.

To evaluate the effect the osmotic agent on the stability of the system over time, a suspension of APAP coated cores was prepared as described above except maltitol was not included in the continuous phase. The amount of APAP solubilized in the continuous phase after 1 week of storage at 30° C. when the continuous phase buffered to pH≥7 lacked maltitol was 4.6 mg/g, while only 0.30 mg/g was detected when the continuous phase buffered to pH≥7 contained 60 wt % maltitol. These data suggest that a suspension which generates a hypertonic condition is necessary to maintain stability of the system over time.

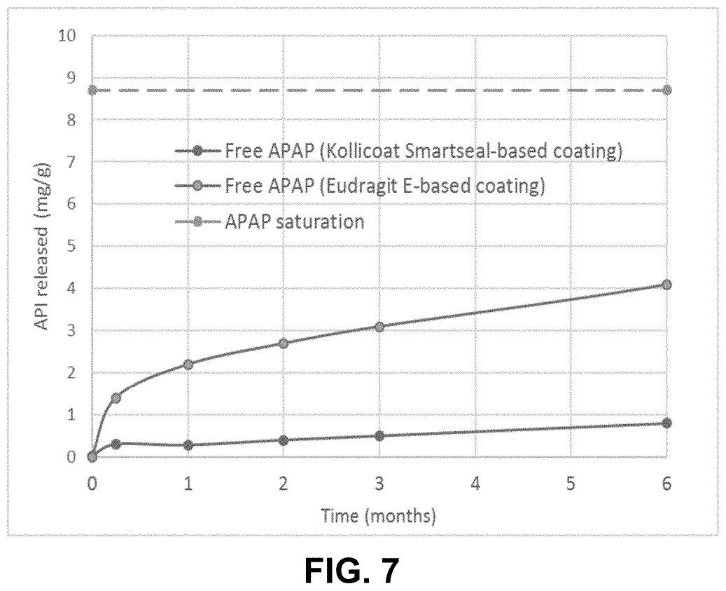

To evaluate whether another gastro soluble polymer could replace KOLLICOAT® Smartseal 30 D, APAP coated cores were prepared as described above except EUDRAGIT® E was used instead of KOLLICOAT® Smartseal 30 D. Suspensions of APAP (Eudragit) coated cores were then prepared and the amount of APAP solubilized in the continuous phase was measured after storage at 30° C., as described above. As shown in FIG. 7, use of EUDRAGIT® E in the outer layer does not impart the same stability characteristics as use of KOLLICOAT® Smartseal 30 D in this experiment.

### Example 2

An immediate release composition was produced that has a stable in vitro dissolution profile after storage of the composition for at least 6 months at 30° C. The composition is a suspension comprising a plurality of Metformin HCl-containing particles dispersed in a continuous phase, the particles consisting of a Metformin HCl-containing core and an outer layer covering the core.

To manufacture the Metformin HCl-containing cores, 1400 g of Metformin HCl was dissolved in 1933 g of water. Drug was layered onto 350 g of Cellet 127 microcrystalline cellulose cores in a Glatt GPC1.1 fluid bed coater using the Wurster process to produce a drug layer of 80% w/w. The layering conditions were controlled at a product temperature of 40° C., air flow of 50-55 m³/h, nozzle pressure of 2.5 bars and mean spray rate of 31 g/min.

An outer layer of a KOLLICOAT® Smartseal 30 D/LUBRITAB® (20 wt %/80 wt %) coating was then applied to the Metformin HCl. 114.3 g of KOLLICOAT® Smartseal 30 D (solid content 30% w/w) and 137.1 g of hydrogenated cottonseed oil (LUBRITAB®) was dissolved in isopropanol (1462.9 g) heated at 78° C. The film was coated onto 400 g of previously prepared Metformin HCl cores in a Glatt GPC1.1 fluid bed coater using the Wurster process to produce a coating ratio of 30 wt %. The coating conditions were controlled at product temperature of 40° C., air flow of 50 m³/h, nozzle pressure of 1.6 bars and mean spray rate of 10 g/min.

Prior to preparing suspensions of Metformin HCl coated cores, the continuous phase was prepared by manufacturing stock solutions of maltitol, xanthan gum, and phosphate buffer. 700.41 g of maltitol was dissolved in 300.08 g of water heated for about 1 hour at 60° C. In a separate container, 4 g of xanthan gum was dissolved in 196.25 g of water under moderate stirring until completely dissolved. A 1.18M phosphate buffer was prepared by solubilizing 16.10 g of KH₂PO₄ in 84 g of water and by incorporating 23.80 g of NaOH. This resulted in a stock solution adjusted at pH 7.53. The continuous phase was prepared in a 2 L beaker by mixing 925.73 g of maltitol stock solution with 108.16 g of xanthan gum stock solution, and 45.85 g of phosphate buffer. The blend was homogenized under gentle stirring for 10-15 minutes at room temperature. The final suspension was prepared by mixing 90 g of the continuous phase with 10 g of the Metformin HCl coated cores. Homogenization was performed under moderate stirring for 5 minutes prior to transfer into a PET bottle. The pH of the suspension was measured to be about pH 7.2. Compositions were stored at 30° C. for further evaluation.

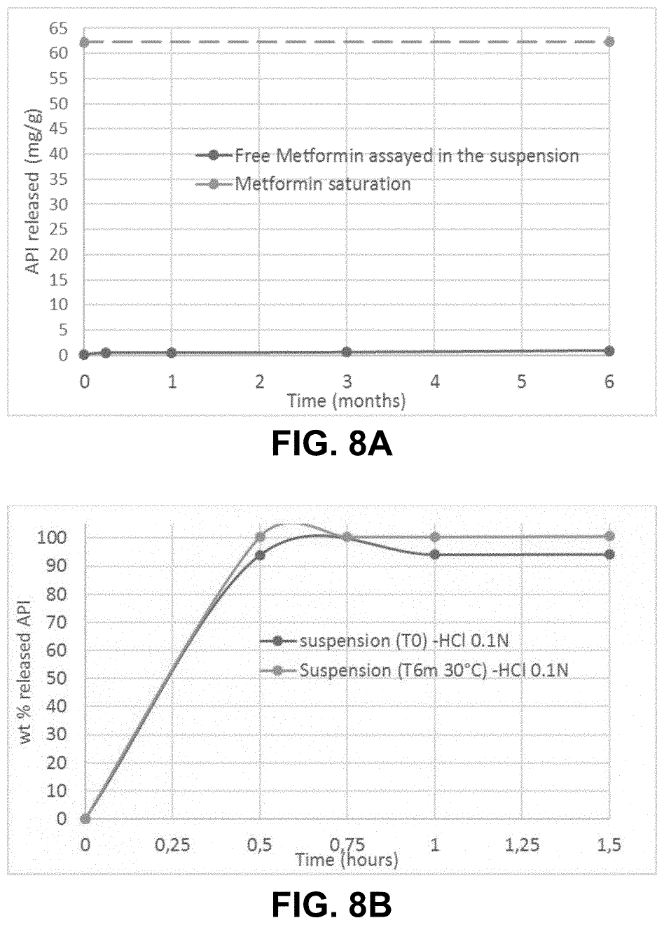

At different times, the stability of the composition was evaluated by measuring Metformin HCl solubilization into the continuous phase and/or the in vitro dissolution profile of the composition after storage and comparing the values to the composition prior to storage and/or to the dry final particles. Briefly, a sample volume of the composition was withdrawn from the container being stored at 30° C. after manual shaking of the container. Dissolution profiles were obtained by introducing a sample volume into a USP Type II apparatus equipped with 1 L vessels filled with 900 mL 0.1N HCl. The vessels were maintained at 37° C. and the rotational speed of the paddles set at 100 RPM. The solubilized Metformin HCl fraction in the continuous phase of the composition was assayed by HPLC after filtering about 3 ml of composition onto a 10 μm filter. As shown in FIG. 8A and FIG. 8B, the composition has stable behavior in suspension for at least 6 months of storage at 30° C. with a solubilized content of Metformin HCl<1 mg/g, thus an amount of Metformin HCl leaching from the particles was reduced by a factor of at least 10 compared to the saturation of the continuous phase by the drug after six months of storage. Bioavailability of the drug is not affected by the coating as the immediate release profile is obtained and maintained overtime.

To evaluate the effect of the osmotic agent on the stability of the system over time, a suspension of Metformin HCl coated cores was prepared as described above except maltitol was not included in the continuous phase. The amount of Metformin HCl solubilized in the continuous phase after 1 week of storage at 30° C. when the continuous phase, buffered to pH≥7 lacked maltitol was 56.8 mg/g, while only 0.45 mg/g was detected when the continuous phase buffered to pH≥7 contained 60 wt % maltitol. These data suggest that a suspension which generates a hypertonic condition is necessary to maintain stability of the system over time.

### Example 3

An immediate release composition was produced that is stable after storage for 1 month at 30° C. The composition is a suspension comprising a plurality of guaifenesin-containing particles dispersed in a continuous phase. The particles have a guaifenesin-containing core and an immediate release coating over the core.

To manufacture the guaifenesin-containing cores, 1140 g of guaifenesin and 60 g of hydroxypropylcelullose (Klucel EF) were dissolved in water (1088 g). Drug was layered onto 300 g of Cellet 127 microcrystalline cellulose cores in a Glatt GPC1.1 fluid bed coater using the Wurster process to produce a drug layer of 80% w/w. The layering conditions were controlled at a product temperature of 30° C., air flow of 50 m³/h, nozzle pressure of 3 bars and mean spray rate of 19 g/min.

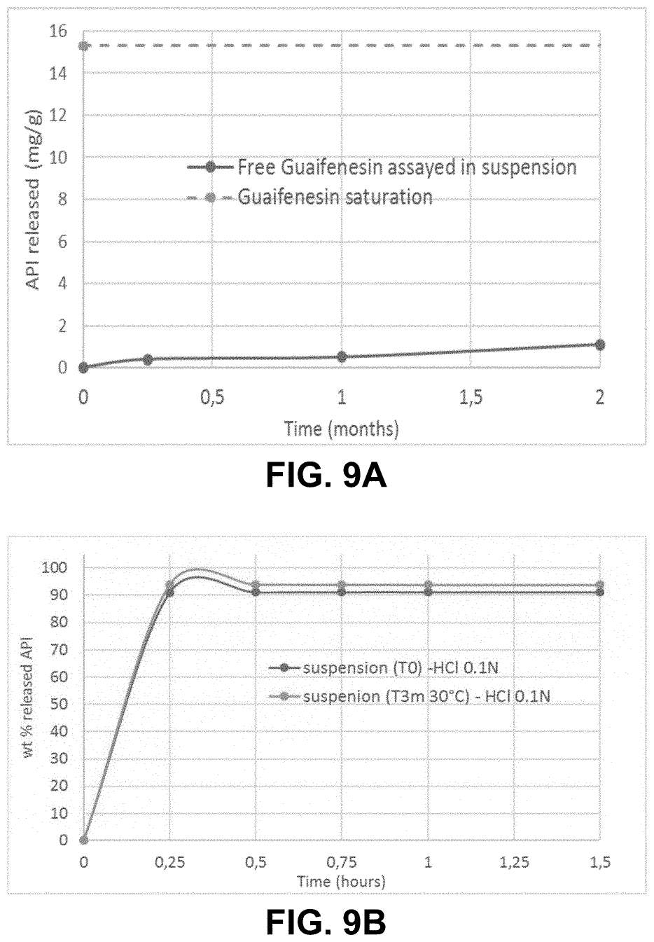

The guaifenesin cores were then coated with an immediate release coating. Briefly, an outer layer of a KOLLICOAT® Smartseal 30 D/LUBRITAB® (20 wt %/80 wt % was then applied to the guaifenesin SR-coated cores to produce the final particle, as generally described in Examples 1 and 2. Suspensions of the final guaifenesin particles were prepared as generally described in Examples 1 and 2 to produce a composition containing 10 wt % (FIGS. 9A and 9B) of the final particles in a continuous phase containing 60 wt % maltitol and 0.2 wt % xanthan gum, buffered to about pH 7.2 with KH₂PO₄.

At different times, the stability of the composition was evaluated by measuring guaifenesin solubilization into the continuous phase and/or the in vitro dissolution profile of the composition after storage, and comparing the values to the composition prior to storage and/or to the dry final particles. Briefly, a sample volume of the composition was withdrawn from the container being stored at 30° C. after manual shaking of the container. Dissolution profiles were obtained by introducing a sample volume into a USP Type II apparatus equipped with 1 L vessels filled with 900 mL 0.1N HCl. The vessels were maintained at 37° C. and the rotational speed of the paddles set at 100 RPM. The solubilized guaifenesin fraction in the continuous phase of the composition was assayed by HPLC after filtering about 3 ml of composition onto a 10 μm filter. As shown in FIG. 9A and FIG. 9B, the composition has stable behavior in suspension for at least 1 month of storage at 30° C.

The solubilized content of guaifenesin in the continuous phase of the suspension was 0.5 mg/g after one month of storage and 1.1 mg/g after 2 months of storage FIG. 9A shows that the free fraction of guaifenesin after 2 months of storage was reduced by a factor of 10 compared to the saturation of the continuous phase by the drug (15.3 mg/g). FIG. 9B shows that while no leaching of the API was observed in the continuous phase over time, the immediate release profile was maintained over time once dissolution was performed.

### Example 4

Two sustained-release compositions of APAP and guaifenesin were produced that was stable after storage for 1 month at 30° C. The compositions were suspensions comprising a plurality of drug-containing particles dispersed in a continuous phase. The particles have a drug-containing core, a sustained-release coating over the core, and an outer layer covering the sustained-release coating.

The APAP-containing cores and guaifenesin-containing cores were manufactured as described in Examples 1 and 3, respectively. The drug-containing cores were then coated with a sustained-release coating. Briefly, a sustained release layer of Ethylcellulose/Polyvinylpyrrolidone/Castor Oil (76 wt %/9 wt %/15 wt %) was applied to the APAP cores to produce the sustained release particle. 97 g of Ethylcellulose, 12 g of Polyvinylpyrrolidone and 19 g of Castor Oil were dissolved in a solvent mixture of Acetone/IPA/water (54 wt %/36 wt %/10 wt %). The film was coated onto 300 g of previously prepared APAP cores in a Glatt GPC1.1 fluid bed coater using the Wurster process to produce a coating ratio of 30 wt %. The coating conditions were controlled at product temperature of 40° C., air flow of 50 m3/h, nozzle pressure of 2.8 bars and mean spray rate of 15 g/min. A sustained release layer of Ethylcellulose/Polyvinylpyrrolidone/Kolliphor RH40/Castor Oil (89 wt %/3 wt %/4 wt %/4 wt %) was applied to the guaifenesin cores to produce the sustained release particle. 78 g of Ethylcellulose, 2.5 g of Polyvinylpyrrolidone, 3.5 g of Kolliphor RH40/3.5 g of Castor Oil were dissolved in 1372 g of a solvent mixture of Acetone/IPA/water (54 wt %/36 wt %/10 wt %). The film was coated onto 350 g of previously prepared guaifenesin cores in a Glatt GPC1.1 fluid bed coater using the Wurster process to produce a coating ratio of 30 wt %. The coating conditions were controlled at product temperature of 41° C., air flow of 50 m3/h, nozzle pressure of 3 bars and mean spray rate of 13 g/min.

An outer layer of a KOLLICOAT® Smartseal 30 D/LUBRITAB® (20 wt %/80 wt %) coating was then applied to the APAP SR-coated cores, as generally described in Example 1. A suspension of the final APAP microparticles was prepared as generally described in Example 1 to produce a composition containing 5 wt % of the final APAP microparticles in a continuous phase containing 60 wt % maltitol and 0.2 wt % xanthan gum, buffered to pH 7.5 with KH₂PO₄.

An outer layer of a KOLLICOAT® Smartseal 30 D/LUBRITAB® (20 wt %/80 wt %) coating was then applied to the guaifenesin SR-coated cores, as generally described in Example 3. A suspension of the final guaifenesin microparticles was prepared as generally described in Example 3 to produce a composition containing 5 wt % of the final guaifenesin microparticles in a continuous phase containing 60 wt % maltitol and 0.2 wt % xanthan gum, buffered to pH 7.5 with KH₂PO₄.

Two sustained-release compositions were produced that have a stable in vitro dissolution profile after storage of the composition at 30° C. The composition is a suspension comprising a plurality of drug-containing particles dispersed in a continuous phase, the particles consisting of a drug-containing core, a sustained-release coating over the core, and an outer layer covering the sustained-release coating. The outer layer is an immediate release coating.

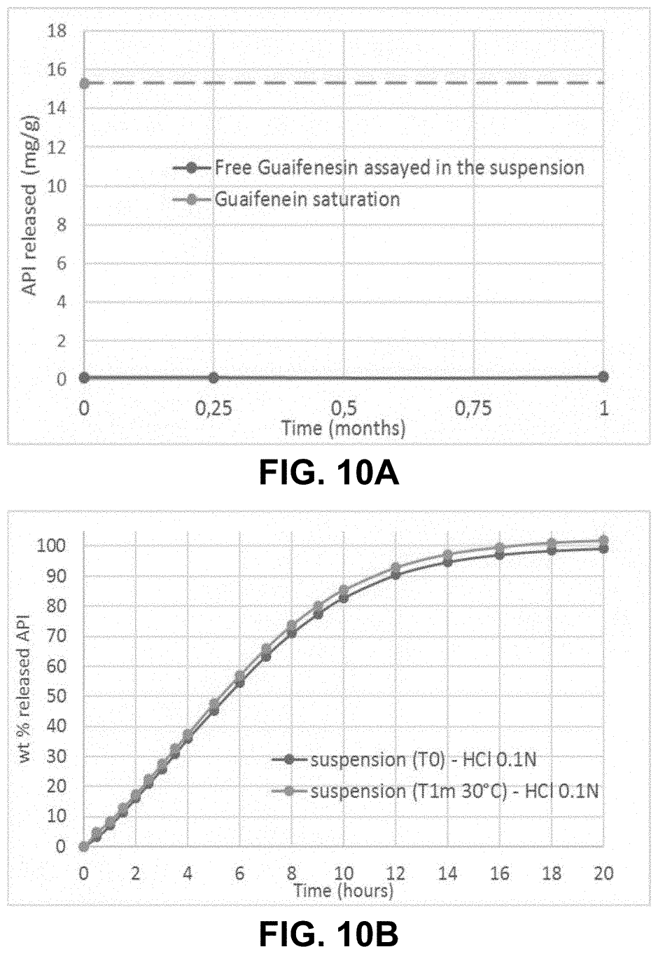

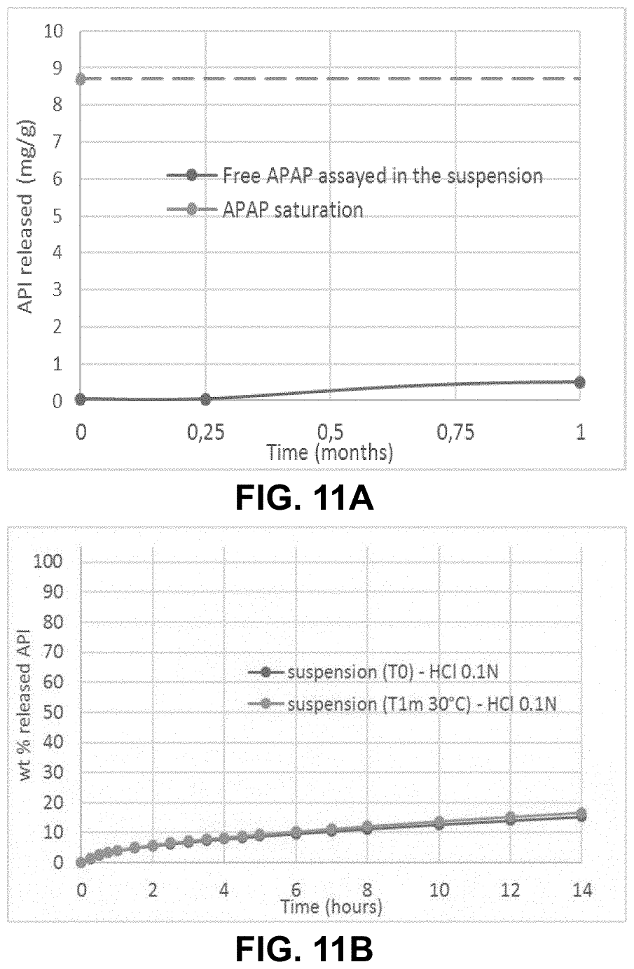

At different time points, the stability of the compositions were evaluated by measuring drug (APAP or guaifenesin) solubilization into the continuous phase and/or the in vitro dissolution profile of the stored composition, and comparing the values to the composition prior to storage and/or to the dry final particles. Briefly, a sample volume of the composition was withdrawn from the container being stored at 30° C. after manual shaking of the container. Dissolution profiles were obtained by introducing a sample volume into a USP Type II apparatus equipped with 1 L vessels filled with 900 mL 0.1N HCl. The vessels were maintained at 37° C. and the rotational speed of the paddles set at 100 RPM. The solubilized drug (APAP or guaifenesin) fraction in the continuous phase of the composition was assayed by HPLC after filtering about 3 ml of composition onto a 10 μm filter. As shown in FIG. 10A, FIG. 10B, FIGS. 11A and 11B, the compositions have stable behavior in suspension for at least 1 month of storage at 30° C. with a solubilized content of drug <1 mg/g. The amount of drug leaching out of the particle in the continuous phase of the suspension was reduced by a factor of 10 compared to the saturation of the continuous phase by the drug.

While no leaching of the API was observed in the continuous phase over time, the sustained release profile was maintained over time once dissolution was performed. The coating of the immediate release coating onto the SR-coated API particle prevents API leaching from the particle to the continuous phase over time while maintaining the dissolution profile characteristics of the particle unchanged.

### Example 5

Examples 7 and 6 demonstrate an osmolality ratio of greater than one is necessary (Example 6) but not sufficient (Example 7) to achieve storage stability. This example describes a method that was developed to determine if a storage-stable suspension can be achieved for a drug formulated with the extended-release coating of Example 6. Briefly, a planar film modeling the extended-release coating was manufactured by film-casting. The planar film was them immersed in an aqueous solution of the drug until the partitioning equilibrium of the drug between the planar film and the solution was reached. The film/water partition coefficient was calculated by a final HPLC titration of the drug solubilized into the film. The partition coefficient (K_{film/water}) between film and water is defined as the ratio of the equilibrium concentrations of the drug solubilized in film and in water. Most frequently it is given as the logarithm to the base 10 (log K_{film/water} the partition coefficient can be written as: K_{film/water}=[drug]_{film} [drug]ₛₒₗᵤₜᵢₒₙ.

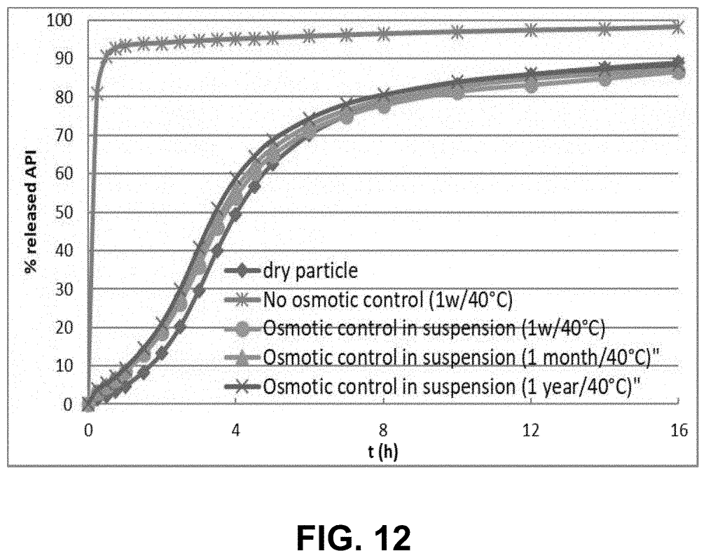

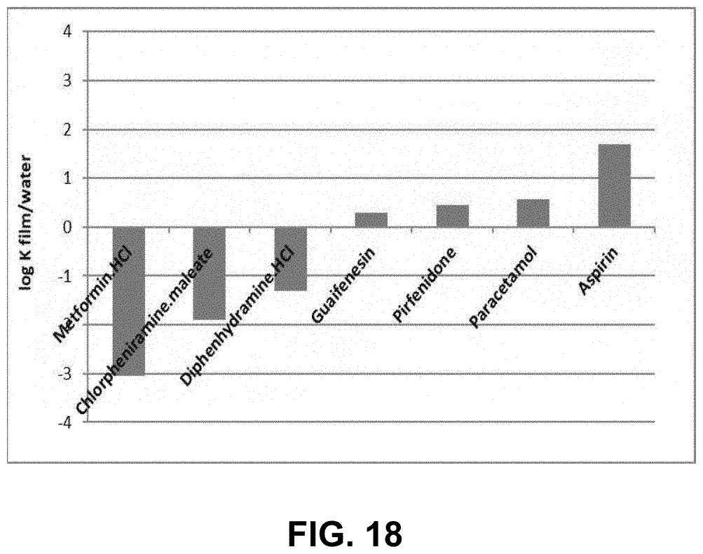

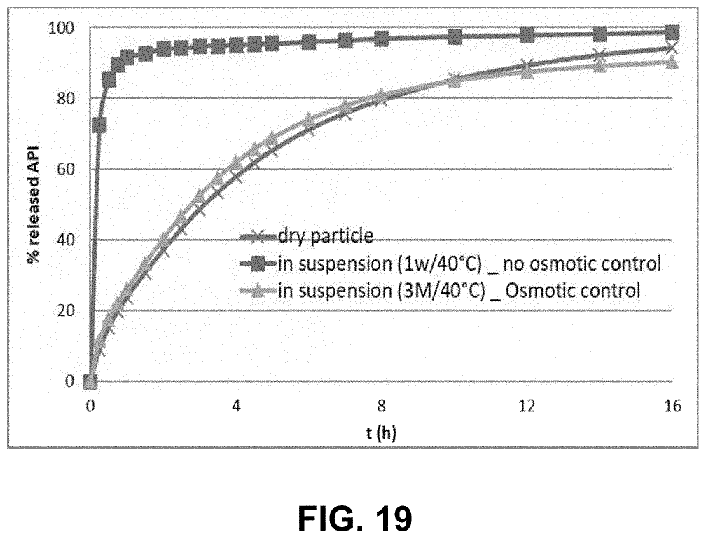

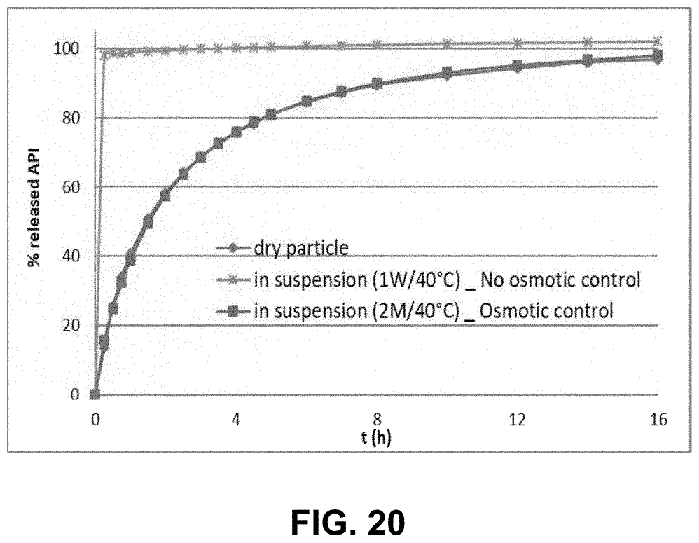

FIG. 18 shows the partition coefficient logarithm for several drugs with an ethylcellulose/polyvinylpyrrolidone/castor oil (80 wt %/10 wt %/10 wt %) planar film, while FIG. 19-20 show in vitro dissolution profiles following storage at 30° C. for two compositions prepared as described in Example 6. The compositions consist of a chlorpheniramine.maleate-containing core (FIG. 19), or a diphenhydramine.HBr-containing core (FIG. 20), and an EC/PVP/CO (80/10/10) coating over the core (coating ratio is 30 wt % for both particles. The continuous phase consisted of 10 wt % particles and 100% Lycasin (osmotic control in FIGS. 19 and 20) or 10 wt % particles and about 0.2 wt % xanthan gum (no osmotic control in FIGS. 19 and 20). Drugs with a negative log K_{film/water} have stable in vitro dissolution profiles following storage for at least 1 month when the osmolality ratio of the composition is greater than 1 (FIGS. 12, 13, 14, 15, 16, 17, 19, and 20). Thus, it can be concluded that storage stable suspensions can be achieved with drugs having a negative log K_{film/water} and an extended-release coating described in Example 6.

### Example 6

This example describes extended-release compositions that have a stable in vitro dissolution profile after storage for months at 30° C. or 40° C. The compositions are suspensions comprising a plurality of Metformin HCl-containing particles dispersed in a continuous phase, the particles consisting of a Metformin HCl-containing core and a coating over the core. The coating contains a film-forming polymer insoluble in the GI tract fluids, a water-soluble polymer, and a plasticizer.

To manufacture the Metformin HCl-containing cores, 1004 g of Metformin HCl was dissolved in 1387 g of water heated at 70° C. Drug was layered onto 300 g of Cellet 127 microcrystalline cellulose cores in a Glatt GPC1.1 fluid bed coater using the Wurster process to produce a drug layer of 77% w/w. The layering conditions were controlled at a product temperature of 40° C., air flow of 50-55 m³/h, nozzle pressure of 2.5 bars and mean spray rate of 25-30 g/min.

An outer layer of an ethylcellulose/polyvinylpyrrolidone/castor oil (80 wt %/10 wt %/10 wt %) coating was then applied to the Metformin HCl cores. The coating solution was prepared by dissolving 196.4 g of Ethocel.STD20, 24.5 g of Plasdone K-29/32, and 24.5 g of castor oil in an acetone/isopropanol/water solvent mixture in mass weight ratios of 54 wt %/36 wt %/10 wt %. The coating solution was applied onto 300 g of previously prepared Metformin HCl cores in a Glatt GPC1.1 fluid bed coater using the Wurster process to produce a coating ratio of 45% w/w. The coating conditions were controlled at product temperature of 40° C., air flow of 50 m³/h, nozzle pressure of 2.8 bars and mean spray rate of 16 g/min.

To prepare the continuous phase, 288 g of maltitol was first dissolved in 123 g of purified water. In a separate container, 3 g of xanthan gum was dissolved under high shear/speed mixing in 147 g of water. The continuous phase was achieved by mixing 83.14 g of the maltitol solution with 4.5 g of the gum solution and 2.4 g of water until uniform. The final suspension was prepared by adding 10 g of the Metformin HCl coated particles to the continuous phase with gentle mixing. Compositions were stored at 30° C. for further evaluation.

At different times, the stability of the composition was evaluated by measuring Metformin HCl solubilization into the continuous phase and/or the dissolution profile of the composition after storage, and comparing the values to the composition prior to storage and/or to the dry final particles. Briefly, after manual shaking a sample volume of the composition was withdrawn from the container being stored. The solubilized drug fraction in the continuous phase of the composition was assayed by HPLC after filtering about 3 ml of composition onto a 10 μm filter. Dissolution profiles were obtained by introducing a sample volume into a USP Type II apparatus equipped with 1 L vessels filled with 900 mL of 50 mM phosphate buffer (PB), pH 6.8. As shown in FIG. 12, the composition is stable for 12 months storage at 40° C. Similar results were obtained when the drug-containing core was core of crystalline Metformin HCl rather than Metformin HCl coated over an inert cellulosic core.

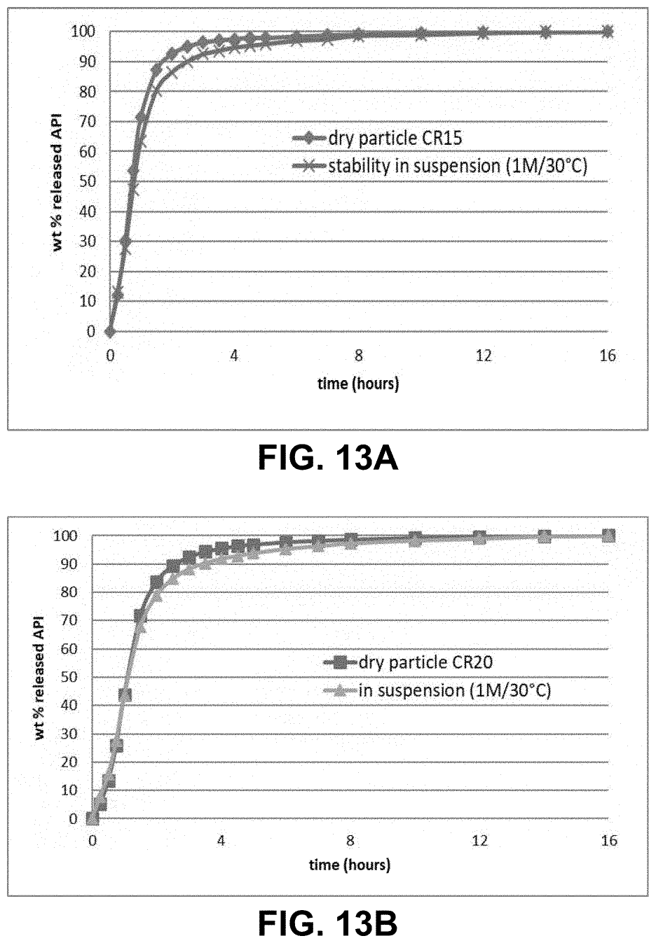

To evaluate the effect of the coating's thickness, Metformin HCl coated particles were prepared as described above with the exception that a coating ratio of 15 wt %, 20 wt %, 25 wt % or 30 wt % was used. Suspensions were prepared and dissolution profiles obtained, both as described above, following storage at 30° C. for one month. As shown in FIG. 13A, coating ratios as low as 15 wt % produced particles with stable behavior in suspension for 1 month storage at 30° C. As shown in FIG. 13B, coating ratios as low as 20 wt % produced particles with stable behavior in suspension for 1 month storage at 30° C.

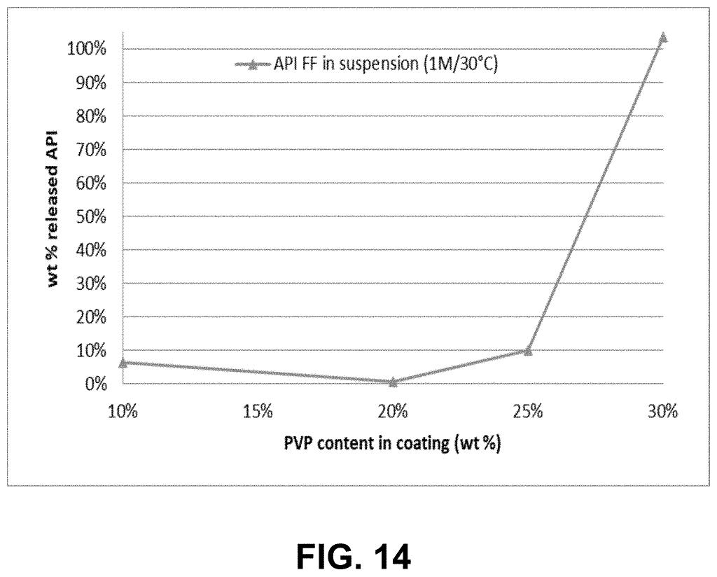

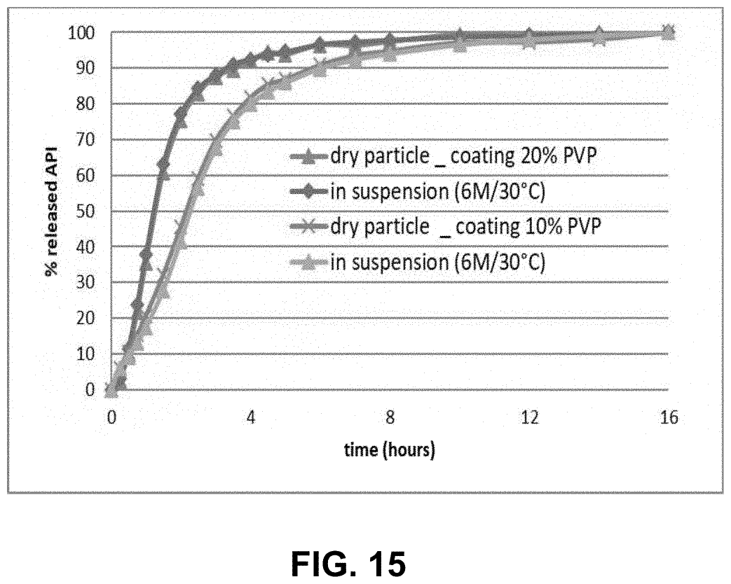

To evaluate the effect of the coating's composition, the amounts of ethylcellulose and polyvinylpyrrolidone in the outer layer were varied. Specifically, coatings of ethylcellulose/polyvinylpyrrolidone/castor oil in amounts of 60 wt %/30 wt %/10 wt %, 65 wt %/25 wt %/10 wt %, and 70 wt %/20 wt %/10 wt % were evaluated. Metformin HCl coated particles and suspensions of the particles were prepared, stored at 30° C., and dissolution profiles obtained as otherwise described above. As shown in FIG. 14, at concentrations of polyvinylpyrrolidone above 20 wt % in the coating, the particles are not stable in suspension, as evidenced by the detection of Metformin HCl in the continuous phase of the suspension after 1 month of storage at 30° C. FIG. 15 illustrates that extended-release particles containing more than 70 wt % of a film-forming polymer and 20 wt % or less of a hydrophilic polymer in the coating have a stable in vitro dissolution profile when stored at 30° C. for at least 6 months.

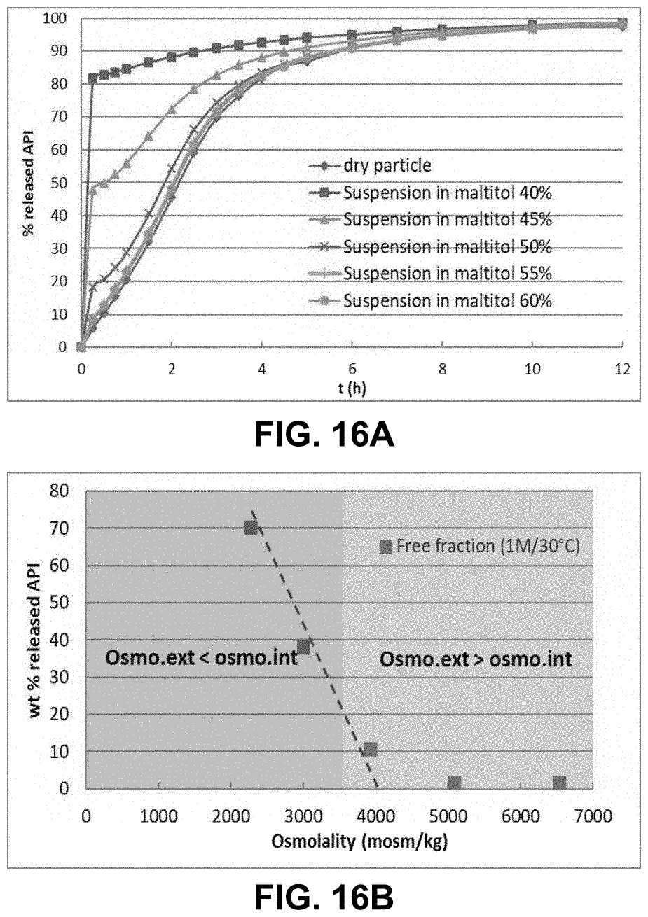

The stability of Metformin HCl particles that have an ethylcellulose/polyvinylpyrrolidone/castor oil (80 wt %/10 wt %/10 wt %) coating were evaluated after 1 month of storage at 30° C. as a suspension in a continuous phase containing different concentrations of maltitol. Metformin HCl coated particles and suspensions of the particles were prepared, stored at 30° C., and Metformin HCl solubilization into the liquid phase was evaluated as generally described above with the exception of the changes described for the composition of the liquid phase. As shown in FIG. 16A and FIG. 16B, complete stabilization of the composition is reached when the external osmolality becomes higher than the osmolality of a Metformin HCl saturated solution (i.e., the osmolality ratio of the composition is greater than 1). Similar data were obtained when other osmotic agents were tested (e.g., citrate, polydextrose, fructose, glucose, maltose, sucrose, erythritol, sorbitol, xylitol, etc.).

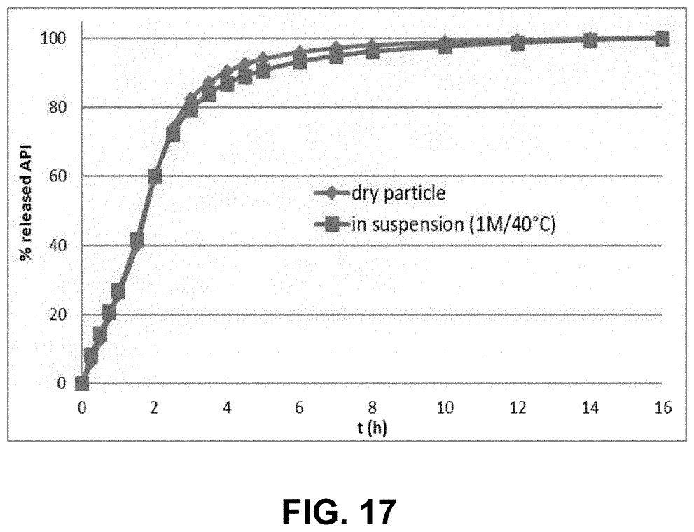

The suitability of other film-forming polymers and water-soluble polymers were also evaluated. Metformin HCl coated particles and suspensions of the particles were prepared, stored at 40° C., and dissolution profiles obtained as generally described above with the exception of the changes described for the composition of the coating and/or continuous phase. FIG. 17 shows polyvinylpyrrolidone can be replaced with copovidone (Plasdone S-630) so the Metformin HCl coated particles have an outer layer of EC-copovidone-CO (80/10/10) and are stable in suspension up to 1 month at 40° C.

### Example 7

The experiments described below evaluated compositions comprising coated drug cores of acetaminophen (APAP), aspirin, and Metformin HCl in a continuous phase containing an osmotic agent and a suspending agent. Numerous unsuccessful attempts were made to produce an extended-release composition consisting of drug-containing particles in a continuous phase where the composition has an in vitro dissolution release profile which upon storage for at least 7 days remains substantially similar (variation of up to +/−15% from the average value) to the initial in vitro dissolution release profile of the particle. In these attempts, the osmolality ratio of the composition was greater than 1 (measured at ambient temperature using a vapor pressure osmometer (model Vapro 5600 XR from ELITech/WESCOR) with a measurement range from 20 mosmol/kg to 3500 mosmol/kg).

To determine the osmolality of the internal phase of the compositions in this example, it was assumed that the osmolality of the internal phase was equivalent to the osmolality of a saturated aqueous solution of a drug at the temperature of interest because the drug-containing cores under evaluation generally contained only a low content of polymeric binder which contributes a negligible osmolality to the internal phase. Osmolality measurements were performed for solutions of acetaminophen, aspirin, or Metformin HCl in water (Table 1). While Metformin HCl has a high osmolality, other APIs such as acetaminophen, pirfenidone or aspirin are poorly to sparingly soluble and consequently have low osmolalities.

TABLE 1
Osmolality of saturated solutions of various drugs.

| | Solubility (mg/g) | | Osmolality (mosmol/kg) | |
|---|---|---|---|---|
| | 25° C. | 40° C. | 25° C. | 40° C. |
| Metformin HCl | 278 | 346 | 3354 | 4216 |
| Acetaminophen | 14 | 20 | 74 | 112 |
| Pirfenidone | 18 | np | 84 | Np |
| Aspirin | 2.5 | np | np | Np |

To determine the osmolality of the external phase of the compositions, it was assumed that only the osmotic agent contributed to the external phase's osmolality because, in addition to the osmotic agent, the external phase contained only minimal amounts of the suspending agent (e.g., 0.1-0.2 wt %). Measurements were obtained experimentally up to 40 wt % sorbitol and up to 50 wt % maltitol and then extrapolated to 60 wt % using a fit with a third order polynomial form (Table 2). Measurements were also obtained for a commercially available sorbitol solution, NEOSORB® 70/70B, containing 70% of dry substance and a minimum of 74% sorbitol in the dry substance (Table 2).

TABLE 2

| | maltitol | | Sorbitol | | Neosorb ® 70/70B | |
|---|---|---|---|---|---|---|
| Dry matter | mOsm/kg sol | mOsm/kg | mOsm/kg sol | mOsm/kg | mOsm/kg sol | mOsm/kg |
| content (wt %) | (experimental) | (extrapolated) | (experimental) | (extrapolated) | (experimental) | (extrapolated) |
| 5% | | | 276 | 269 | | |
| 10% | 333 | 330 | 599 | 610 | | |
| 15% | | | 954 | 960 | | |
| 20% | 779 | 791 | 1376 | 1364 | 333 | 332 |
| 25% | | | 1844 | 1830 | | |
| 30% | 1340 | 1322 | 2407 | 2426 | 779 | 783 |
| 40% | 2265 | 2277 | 4125 | 4122 | 1340 | 1334 |
| 50% | 3882 | 3879 | | 6816 | 2265 | 2269 |
| 60% | | 6538 | | 10777 | 3882 | 3881 |
| 65% | | | | | | 5171 |
| 70% | | | | | | 6709 |
| 75% | | | | | | 8600 |
| 80% | | | | | | 10887 |

Several conclusions can be drawn from the measurements in Table 1 and 2. First, if greater than 30 wt % maltitol is used as an osmotic agent, the osmolality of the external phase will be higher than the internal phase osmolality of microparticle cores containing APAP, aspirin and pirfenidone, at any temperature from 25° C. to 40° C., and therefore the osmolality ratio will be greater than 1. Second, if greater than 50 wt % maltitol is used as an osmotic agent, the osmolality of the external phase will be greater than the internal phase osmolality of microparticle cores containing Metformin HCl at 25° C., and therefore the osmolality ratio will be greater than 1. Third, if greater than 60 wt % maltitol is used as an osmotic agent, the osmolality ratio will be greater than 1 at temperatures up to 40° C. for each of the four different drugs considered in the internal phase, and therefore the osmolality ratio will be greater than 1. Fourth, if greater than 45 wt % sorbitol or greater than 65% NEOSORB® 70/70B is used as an osmotic agent, then the osmolality ratio will be greater than 1 at temperatures up to 40° C. for each of the four different drugs considered in the internal phase, and therefore the osmolality ratio will be greater than 1.

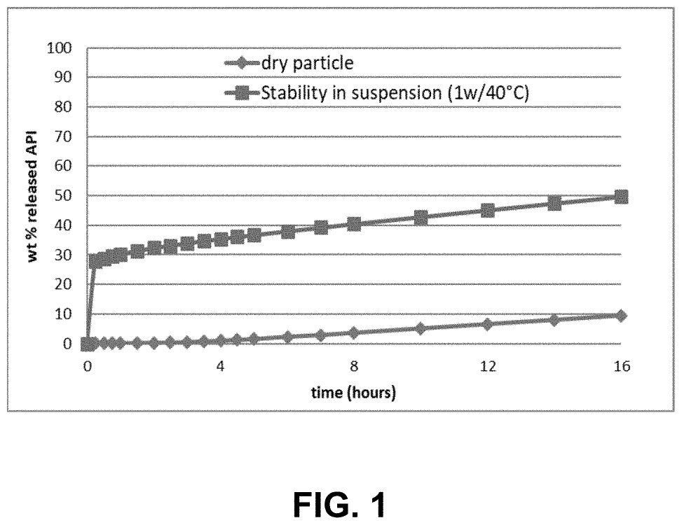

With the above in mind, several experiments were designed to test the stability of compositions comprising coated drug cores of acetaminophen (APAP), aspirin, or Metformin HCl, in a continuous phase containing an osmotic agent and a suspending agent. Briefly, the coated drug cores (i.e., drug-containing particles) were prepared by first spraying a drug solution onto a neutral cellulosic core in fluid bed then followed by spraying a coating solution onto this drug-layered core in fluid bed. The coating weight ratio was 30 wt % (Exp. 2-5) or 50 wt % (Exp. 1). Drug-containing particles for a given drug were dispersed in a continuous phase (10 wt % of the total weight of the suspension), the continuous phase consisting of water, an osmotic agent and a suspending agent. In each experiment, the suspending agent used was xanthan gum in an amount that was about 0.1 wt % to 0.2 wt % of the total weight of continuous phase. Compositions were stored for a period of time under various temperatures, as indicated in Table 3. To evaluate the stability of the stored compositions, the in vitro dissolution profile of the stored composition was compared to the dry particles for reference. Briefly, a sample volume of the composition was withdrawn from the container being stored after manual shaking of the container. Dissolution profiles were obtained by introducing a sample volume into a USP Type II apparatus equipped with 1 L vessels filled with 900 mL 0.1N HCl or phosphate buffer (PB) at pH 6.8. The vessels were maintained at 37° C. and the rotational speed of the paddles set at 100 RPM. The dry particles were directly introduced into the vessel. The results are described in Table 3 and FIG. 1-5. Overall, these compositions had an in vitro dissolution release profile which upon storage for at least 7 days at elevated temperatures (e.g., 30° C. or 40° C.) did not remain stable, despite the fact that the compositions had an osmolality ratio greater than 1.

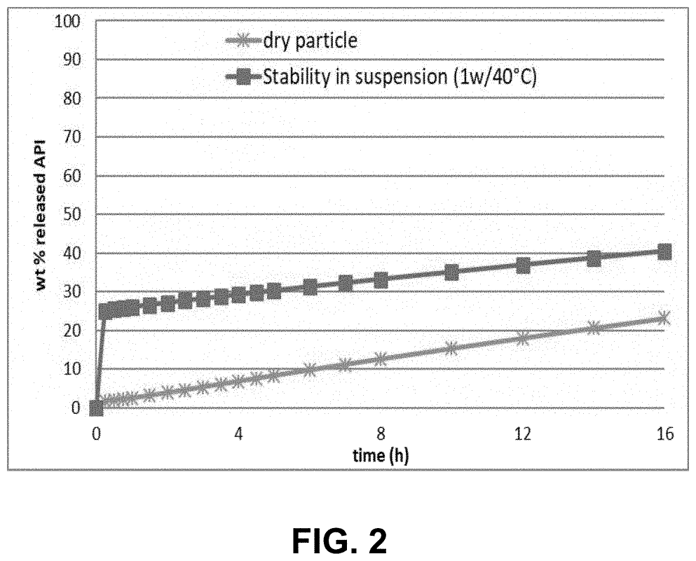

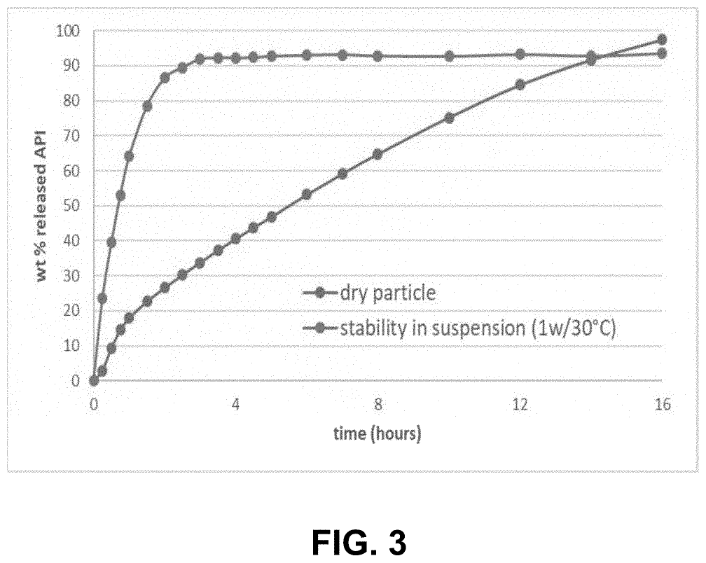

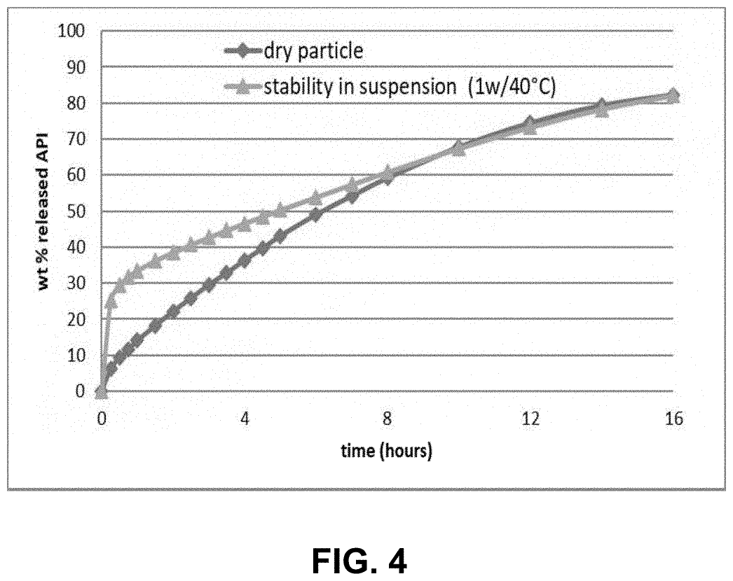

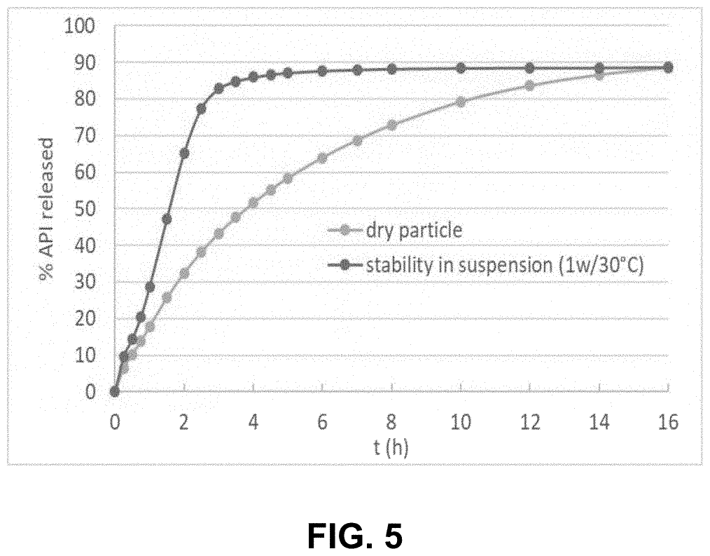

TABLE 3

| | | | | Samples |
|---|---|---|---|---|
| Exp. | Drug-containing particle | | Continuous | Dissolution Test |
| No. | Core | Coating | phase | Conclusions |
| 1 | 20 wt % Cellet 90 | Ratio: 50 wt % | 60 wt % | Dry μP, composition stored for 1 w @ |
| | 72 wt % APAP | 80 wt % EC | maltitol | 40° C. |
| | 8 wt % Klucel | 10 wt % PVP | | USP II, 37° C., PB pH 6.8 |
| | | 10% wt % CO | | The release profile of the composition is |
| | | | | not stable upon storage for 1 week at |
| | | | | 40° C. (FIG. 1). |
| 2 | 60 wt % Cellet 127 | Ratio: 30 wt % | 60 wt % | Dry μP, composition stored for 1 w @ |
| | 36 wt % APAP | 90 wt % EC | maltitol | 40° C. |
| | 4 wt % Klucel | 10 wt % CO | | USP II, 37° C., PB pH 6.8 |
| | | | | The release profile of the composition is |
| | | | | not stable upon storage for 1 week at |
| | | | | 40° C. (FIG. 2). |
| 3 | 20 wt % Cellet 127 | Ratio: 30 wt % | 60 wt % | Dry μP, composition stored for 1 w @ |
| | 72 wt % APAP | 65 wt % RS | maltitol | 30° C. |
| | 8 wt % Klucel | 25 wt % talc | | USP II, 37° C., PB pH 6.8 |
| | | 10% wt % DBS | | The release profile of the composition is |
| | | | | not stable upon storage for 1 week at |
| | | | | 30° C. (FIG. 3). |
| 4 | 60 wt % Cellet 127 | Ratio: 30 wt % | 80 wt % | Dry μP, composition stored for 1 w @ |
| | 40 wt % aspirin | 80 wt % EC | Neosorb ® | 40° C. |
| | | 10 wt % PVP | 70/70B | USP II, 37° C., PB pH 6.8 |
| | | 10% wt % CO | | The release profile of the composition is |
| | | | | not stable upon storage for 1 week at |
| | | | | 40° C. (FIG. 4). |
| 5 | 20 wt % Cellet 127 | Ratio: 30 wt % | 60 wt % | Dry μP, composition stored for 1 w @ |
| | 80 wt % M HCl | 74 wt % RS | maltitol | 30° C. |
| | | 19 wt % talc | | USP II, 37° C., PB pH 6.8 |
| | | 7% wt % TEC | | The release profile of the composition is |
| | | | | not stable upon storage for 1 week at |
| | | | | 30° C. (FIG. 5). |

Abbreviations:
APAP (acetaminophen),
M HCl (Metformin HCl),
EC (ethylcellulose),
PVP (polyvinylpyrrolidone),
CO (castor oil),
RS (EUDRAGIT ® RS),
DBS (dibutyl sebacate),
TEC (triethyl citrate),
PVAc (polyvinyl Acetate PPG),
μP (drug-containing particle),
w (week),
h (hour)

### Example 8

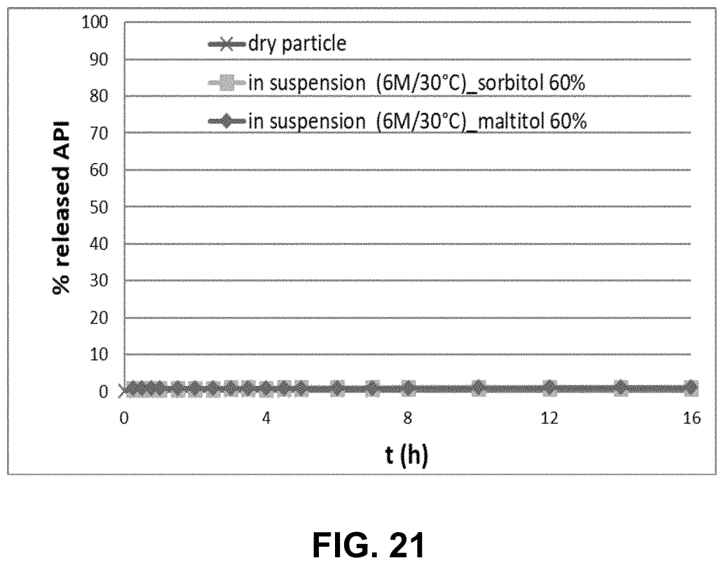

This example describes delayed-release compositions that have a stable in vitro dissolution profile after storage for months at 30° C. in 60% maltitol or 60% sorbitol as seen in FIG. 21. The compositions are suspensions comprising a plurality of paracetamol-containing particles dispersed in a continuous phase, the particles consisting of a paracetamol-containing core and a coating over the core. The coating contains one or more hydrophobic compounds that are crystalline in the solid state and one or more polymers carrying groups that are ionized at neutral pH. Preferably, the ratio of the hydrophobic compound(s) to polymer(s) is about 1.5.

To manufacture the APAP-containing cores, 1080 g of APAP was dissolved in 120 g of hydroxypropylcellulose (KLUCEL EF) in water (1250 g)/ethanol (1250 g) solvent mixture. Drug was layered onto 300 g of Cellet microcrystalline cellulose cores in a Glatt GPC1.1 fluid bed coater using the Wurster process to produce a drug layer of 25% w/w. The layering conditions were controlled at a product temperature of 40° C., air flow of 60 m³/h, nozzle pressure of 2.8 bars and mean spray rate of 25-30 g/min.

An outer layer of a EUDRAGIT® S100/LUBRITAB® coating (40 wt %/60 wt %) was then applied to the APAP cores. The coating solution was prepared by dissolving 46.7 g of EUDRAGIT® S100 (Evonik) and 70 g of hydrogenated cottonseed oil (LUBRITAB®, JRS Pharma) in hot isopropanol. The coating solution was applied onto 350 g of previously prepared APAP cores in a Glatt GPC1.1 fluid bed coater using the Wurster process to produce a coating ratio of 25% w/w. The coating conditions were controlled at product temperature of 40° C., air flow of 70 m³/h, nozzle pressure of 1.6 bars and mean spray rate of 5-9 g/min. A curing step was performed in situ for 2 hours at 55° C. with an air flow of 70 m³/h.

To prepare the continuous phase, 280 g of maltitol or sorbitol was first dissolved in 120 g of purified water, followed by the addition of 4.9 g of citric acid. In a separate container, 1 g of xanthan gum was dissolved under high shear/speed mixing in 50 g of water. 23.2 g of the gum solution was then transferred to the main container, and concentrated NaOH (1.2 g) was added to obtain a pH of 3. Purified water was added to achieve a final weight of 465 g and the solution was mixed until uniform. The final suspension was prepared by adding 51.7 g of the APAP coated particles to the continuous phase with gentle mixing. Compositions were stored at 30° C. for further evaluation.

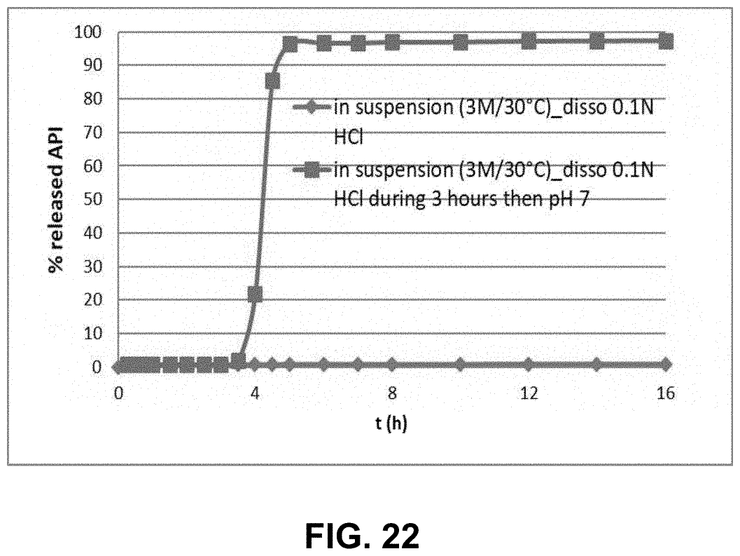

As shown in FIG. 22, the composition still responds to a pH change even after 3 months storage in suspension.

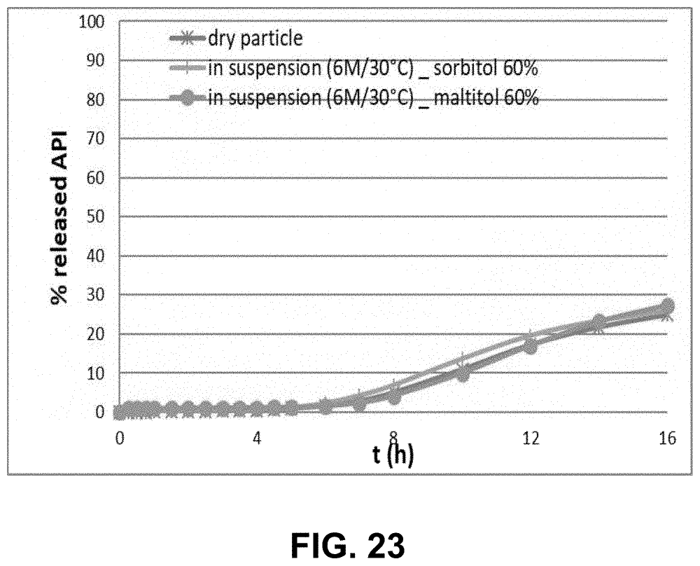

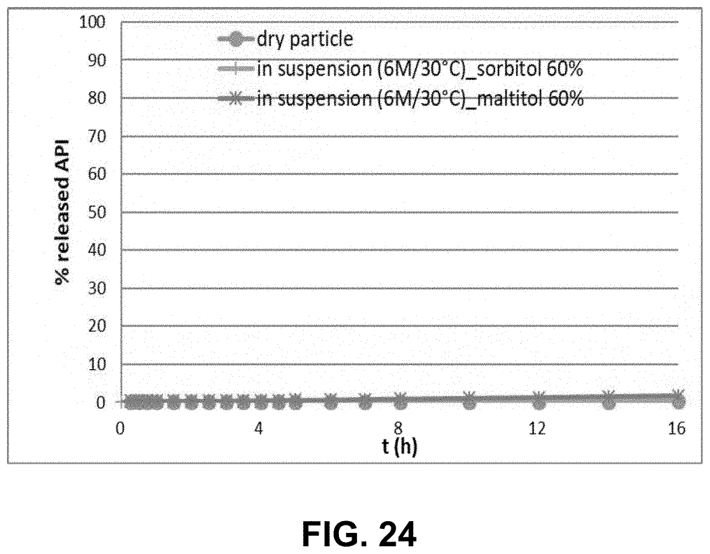

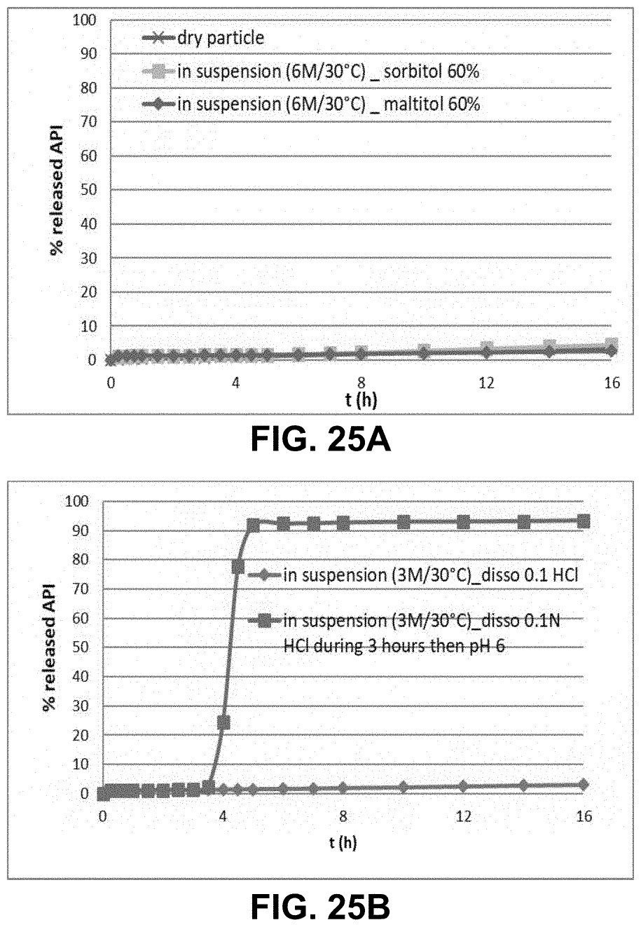

The suitability of polymers other than EUDRAGIT® S100 and osmotic agents other than maltitol were also demonstrated. For example, coatings of EUDRAGIT® L100-55/LUBRITAB® (40 wt %/60 wt %; FIG. 23), EUDRAGIT® S100/EUDRAGIT® L100-55/LUBRITAB® (20 wt %/20 wt %/60 wt %; FIG. 24), and EUDRAGIT® L100/LUBRITAB® (40 wt %/60 wt %; FIG. 25A) were evaluated in continuous phases containing 60 wt % maltitol and/or 60 wt % sorbitol and 0.2 wt % xanthan gum at pH3 (citric acid 50 mM). APAP-coated particles and suspensions of the particles were prepared, stored at 30° C. for 6 months, and dissolution profiles obtained as generally described above with the exception of the changes described for the composition of the coating and/or continuous phase. As shown in FIG. 25B, the composition still responds to a pH change even after 3 months storage in suspension.

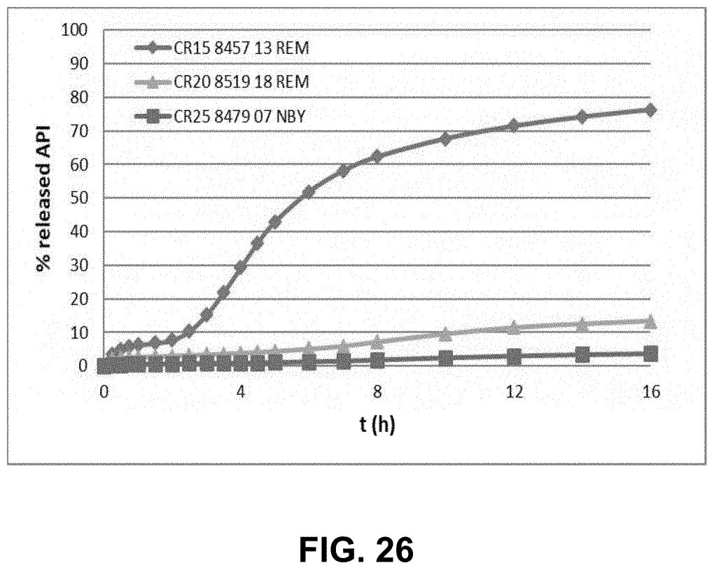

To evaluate the effect of the coating's thickness, paracetamol-coated particles with a EUDRAGIT® L100/LUBRITAB® (40 wt %/60 wt %) coating were prepared as described above with the exception that a coating ratio of 15 wt % or 20 wt % was used. As shown in FIG. 26, a coating ratio of 20 wt % or greater produced particles with stable behavior in suspension.

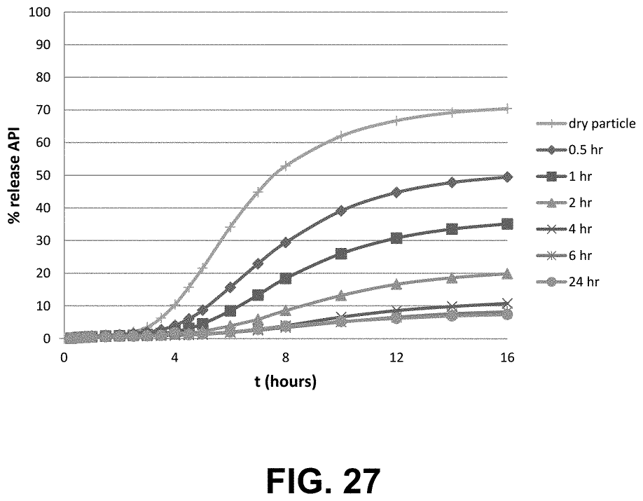

The curing step can be performed at high temperature (about 40° C. to about 60° C.) and high humidity (75-95% RH) for an amount of time until the cured coated particles have a slower in vitro dissolution profile and a constant similarity factor (f2) compared to the uncured coated particles (USP dissolution apparatus 2 (paddle), 900 mL, 37 C, 100 rpm, HCl, pH 1.4). See, for example, FIG. 27.

### Example 9

Several experiments were designed to test the stability of compositions comprising coated drug cores of acetaminophen (APAP), Metformin HCl, guaifenesin, or Chlorpheniramine.maleate in a continuous phase containing an osmotic agent. Briefly, the coated drug cores (i.e., drug-containing particles) were prepared by spraying a water or water/ethanol solution containing the drug and binder onto Cellet 127 microcrystalline cellulose. This step was performed using a Glatt GPC1.1 fluid bed coater using the Wurster process to produce a drug layer with 80-85 wt % ratio. An outer layer of a Eudragit L100/Lubritab coating was then applied onto the drug-containing particles to produce the final particle. Eudragit L100 and Lubritab were dissolved in isopropanol heated at 78° C. and the solution applied onto previously prepared drug-containing particles in a Glatt GPC1.1 fluid bed coater using the Wurster process to produce a coating ratio of 25 wt %. The coating conditions were controlled at product temperature of 41° C., air flow of 50 m3/h, nozzle pressure of 1.6 bars and mean spray rate of 10 g/min. Drug-containing particles for a given drug were dispersed in a continuous phase (10 wt % of the total weight of the suspension), the continuous phase consisting of water, an osmotic agent, and 0.2 wt % xanthan gum and adjusted to pH3 (citric acid 50 mM).

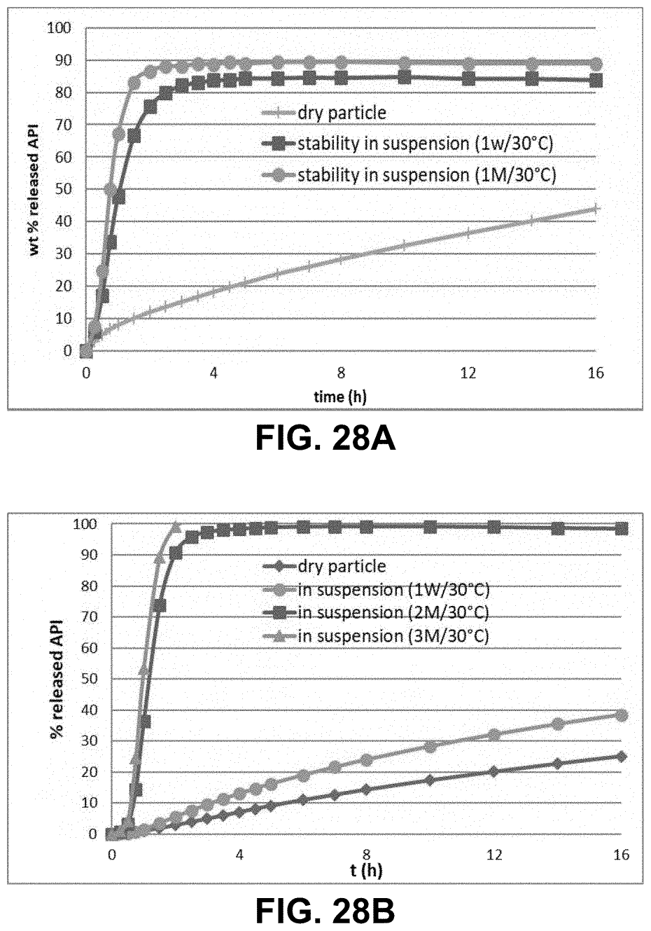

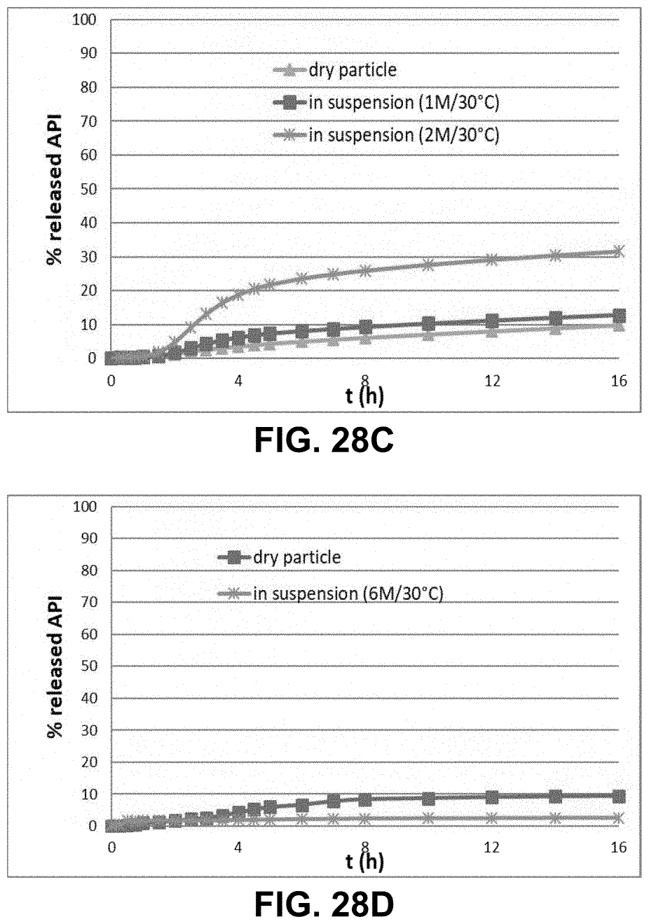

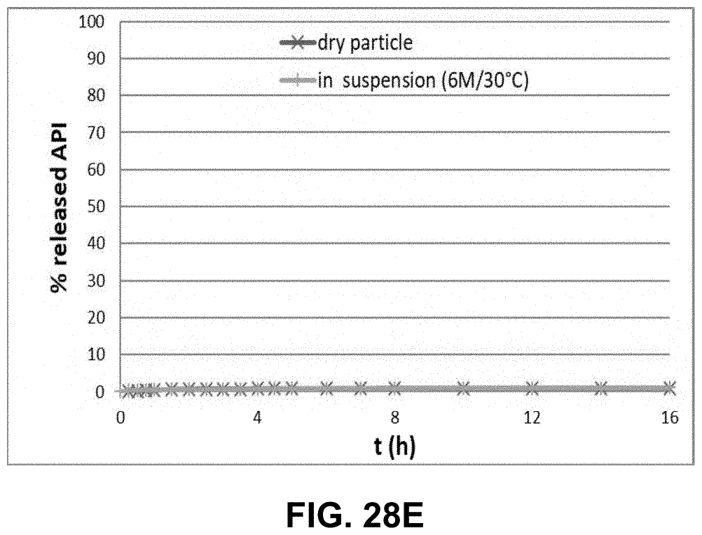

Compositions were stored for various periods of time at 30° C., as indicated in Table 4. To evaluate the stability of the stored compositions, the in vitro dissolution profile of the stored composition was compared to the initial dissolution profile of the composition and/or the dry particle. Briefly, a sample volume of the composition was withdrawn from the container being stored after manual shaking of the container. Dissolution profiles were obtained by introducing a sample volume into a USP Type II apparatus equipped with 1 L vessels filled with 900 mL 0.1N HCl. The vessels were maintained at 37° C. and the rotational speed of the paddles set at 100 RPM. The results are described in Table 4 and FIGS. 28A-28E and 30-33. Overall, the compositions of exp. 6, 7, and 8 had an in vitro dissolution release profile which upon storage for at least 2 months at 30° C. did not remain stable, while the compositions of exp. 9-14 had in vitro dissolution release profiles which remained stable upon storage for up to 6 months at 30° C.

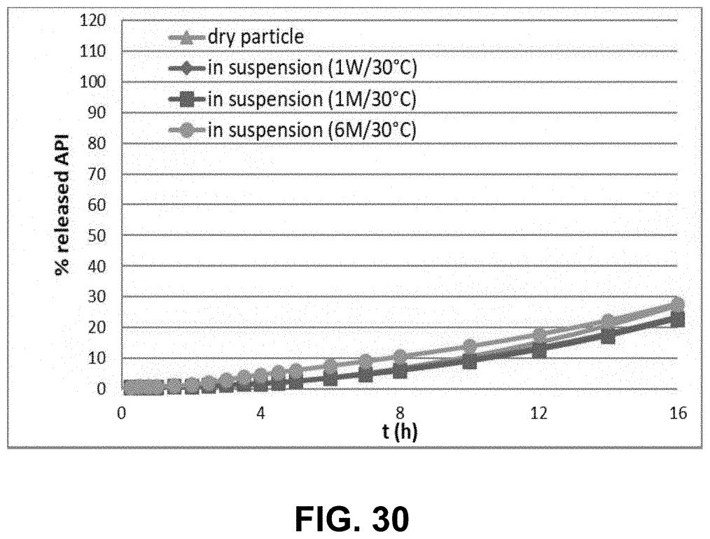

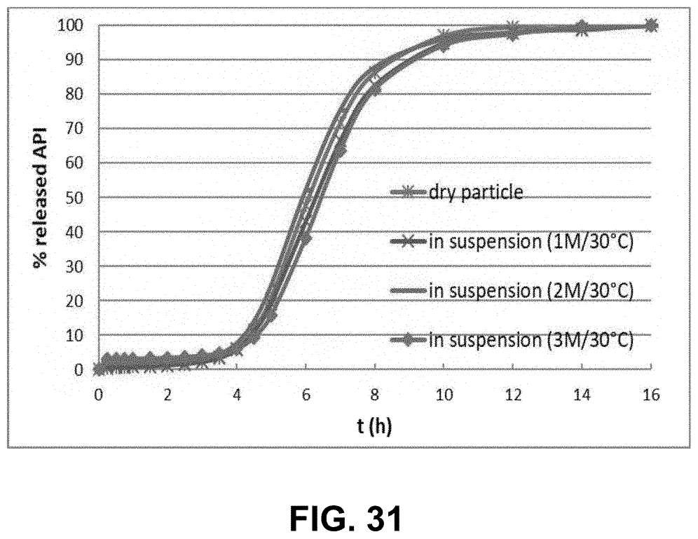

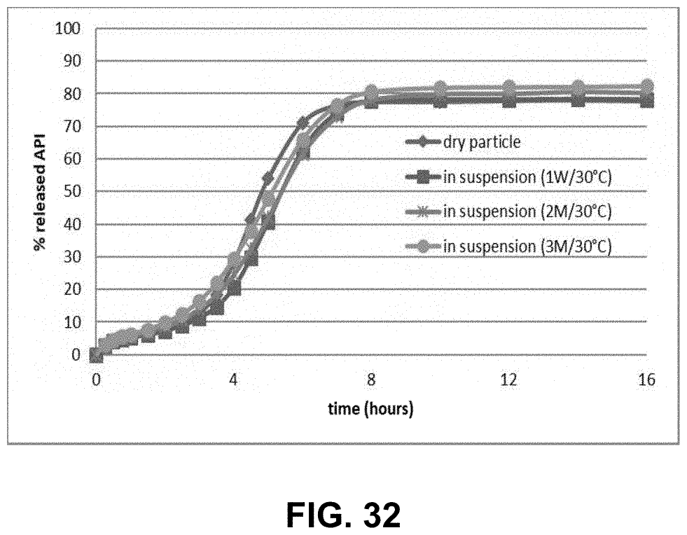

TABLE 4

| | | | | Samples |
|---|---|---|---|---|
| Exp. | Drug-containing particle | | Continuous | Dissolution Test |
| No. | Core | Coating | phase | Conclusions |
| 6 | 20 wt % Cellet 127 | Ratio: 25 wt % | 60 wt % | Dry μP, composition stored for 1 w or 1 m |
| | 72 wt % APAP | 100% | maltitol | @ 30° C. |
| | 8 wt % Klucel | EUDRAGIT ® | 0.2 wt % | USP II, 37° C., 0.1N HCl |
| | | L100 | xanthan | The release profile of the composition is |
| | | | (pH 3) | not stable upon storage for 1 week at |
| | | | | 30° C. (FIG. 28A). |
| 7 | 20 wt % Cellet 127 | Ratio: 25 wt % | 60 wt % | Dry μP, composition stored for 1 w, 2 m |
| | 72 wt % APAP | 80% | maltitol | or 3 m @ 30° C. |
| | 8 wt % Klucel | EUDRAGIT ® | 0.2 wt % | USP II, 37° C., 0.1N HCl |
| | | L100 | xanthan | The release profile of the composition is |
| | | 20% Lubritab | (pH 3) | not stable upon storage for 1 week at |
| | | | | 30° C. (FIG. 28B). |
| 8 | 20 wt % Cellet 127 | Ratio: 25 wt % | 60 wt % | Dry μP, composition stored for 1 m or 2 m |
| | 72 wt % APAP | 60% | maltitol | @ 30° C. |
| | 8 wt % Klucel | EUDRAGIT ® | 0.2 wt % | USP II, 37° C., 0.1N HCl |
| | | L100 | xanthan | The release profile of the composition is |
| | | 40% Lubritab | (pH 3) | not stable upon storage for 2 months at |
| | | | | 30° C. (FIG. 28C). |
| 9 | 20 wt % Cellet 127 | Ratio: 25 wt % | 60 wt % | Dry μP, composition stored for 6 m @ |
| | 72 wt % APAP | 40% | maltitol | 30° C. |
| | 8 wt % Klucel | EUDRAGIT ® | 0.2 wt % | USP II, 37° C., 0.1N HCl |
| | | L100 | xanthan | The release profile of the composition is |
| | | 60% Lubritab | (pH 3) | stable upon storage for 6 months at 30° C. |
| | | | | (FIG. 28D). |
| 10 | 20 wt % Cellet 127 | Ratio: 25 wt % | 60 wt % | Dry μP, composition stored for 6m @ |
| | 72 wt % APAP | 20% | maltitol | 30° C. |
| | 8 wt % Klucel | EUDRAGIT ® | 0.2 wt % | USP II, 37° C., 0.1N HCl |
| | | L100 | xanthan | The release profile of the composition is |
| | | 80% Lubritab | (pH 3) | stable upon storage for 6 months at 30° C. |
| | | | | (FIG. 28E). |
| 11 | 20 wt % Cellet 127 | Ratio: 25 wt % | 60 wt % | Dry μP, composition stored for 1 w, 1 m, |
| | 80 wt % Metformin | 40% | sorbitol | or 6 m @ 30° C. |
| | HCl | EUDRAGIT ® | 0.2 wt % | USP II, 37° C., 0.1N HCl |
| | | L100 | xanthan | The release profile of the composition is |
| | | 60% Lubritab | (pH 3) | stable upon storage for up to 6 months at |
| | | | | 30° C. (FIG. 30). |
| 12 | 15 wt % Cellet 127 | Ratio: 25 wt % | 60 wt % | Dry μP, composition stored for 1 m, 2 m, |
| | 81 wt % | 40% | maltitol | or 3 m @ 30° C. |
| | Guaifenesin | EUDRAGIT ® | 0.2 wt % | USP II, 37° C., 0.1N HCl |
| | 4 wt % Klucel | L100 | xanthan | The release profile of the composition is |
| | | 60% Lubritab | (pH 3) | stable upon storage for up to 3 months at |
| | | | | 30° C. (FIG. 31). |
| 13 | 20 wt % Cellet 127 | Ratio: 25 wt % | 60 wt % | Dry μP, composition stored for 1 w, 2 m, |
| | 76 wt % | 40% | sorbitol | or 3 m @ 30° C. |
| | Chlorpheniramine | EUDRAGIT ® | 0.2 wt % | USP II, 37° C., 0.1N HCl |
| | maleate | L100 | xanthan | The release profile of the composition is |
| | 4 wt % Klucel | 60% Lubritab | (pH 3) | stable upon storage for up to 3 months at |
| | | | | 30° C. (FIG. 32). |

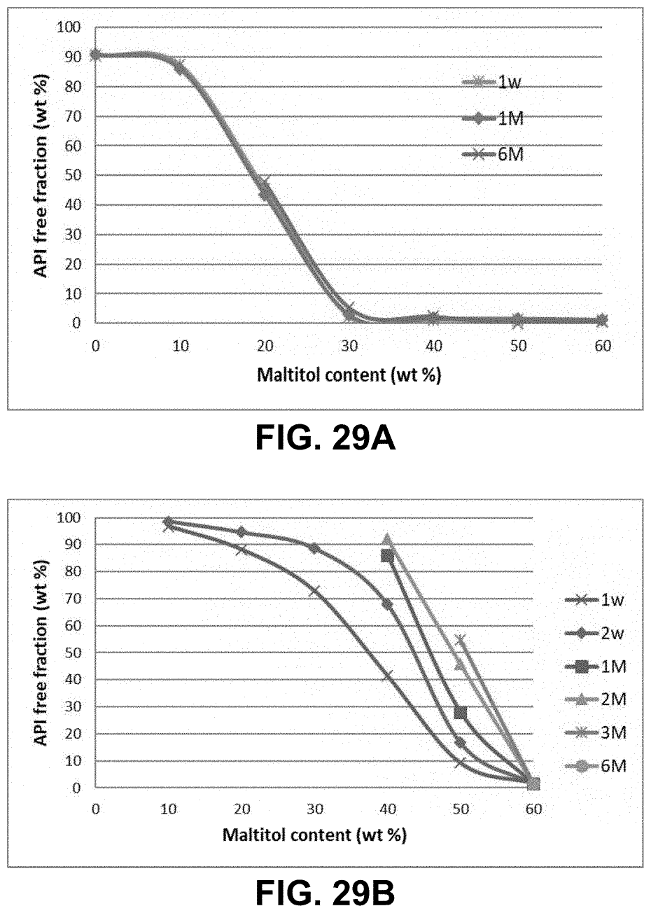

To determine the osmolality of the external phase of the compositions, it was assumed that only the osmotic agent contributed to the external phase's osmolality because, in addition to the osmotic agent, the external phase contained only minimal amounts of the suspending agent (e.g., 0.1-0.2 wt % xanthan). Measurements were obtained experimentally up to 60 wt % maltitol (FIG. 29A, FIG. 29B, FIG. 31) or 60 wt % sorbitol (FIG. 30 and FIG. 32).

Several conclusions can be drawn from the results in FIG. 29A and FIG. 29B. First, if greater than 30 wt % maltitol is used as an osmotic agent, the osmolality of the continuous phase will be higher than the internal phase osmolality of microparticle cores containing APAP and higher than 1300 mosm/kg. If greater than 60 wt % maltitol is used as an osmotic agent, the osmolality of the continuous phase will be higher than the internal phase osmolality of microparticle cores containing Metformin HCl and higher than 1300 mosm/kg at 30° C. Second, FIG. 29A shows stability of APAP particles coated with Eudragit L100-Lubritab (40/60) as a function of external osmolality. Stability was obtained when osmolality of the continuous phase was higher than a saturated API solution and with a minimum of 1300 mOsm/kg (i.e., equivalent to maltitol 30% in FIG. 29A). Third, FIG. 29B shows stability of Metformin HCl particles coated with Eudragit L100-Lubritab (40/60) as a function of external osmolality. Stability was obtained when osmolality of the continuous phase was higher than a saturated API solution (i.e. 6500 mOsm/kg equivalent to maltitol 60% in FIG. 29B) and with a minimum of 1300 mOsm/kg.

### Example 10

This example describes compositions that have a stable in vitro dissolution profile after storage for months at 30° C. when stored as a suspension under osmotic control. The compositions are suspensions comprising a plurality of drug-containing particles dispersed in a continuous phase, the particles consisting of a drug-containing core and a coating over the core.

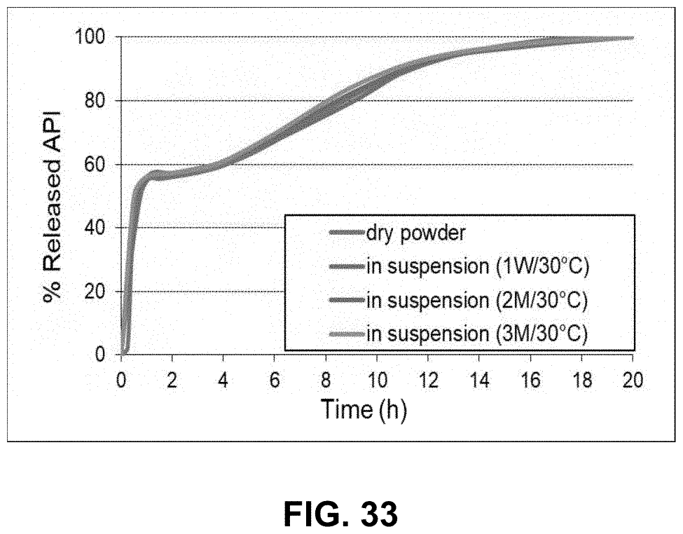

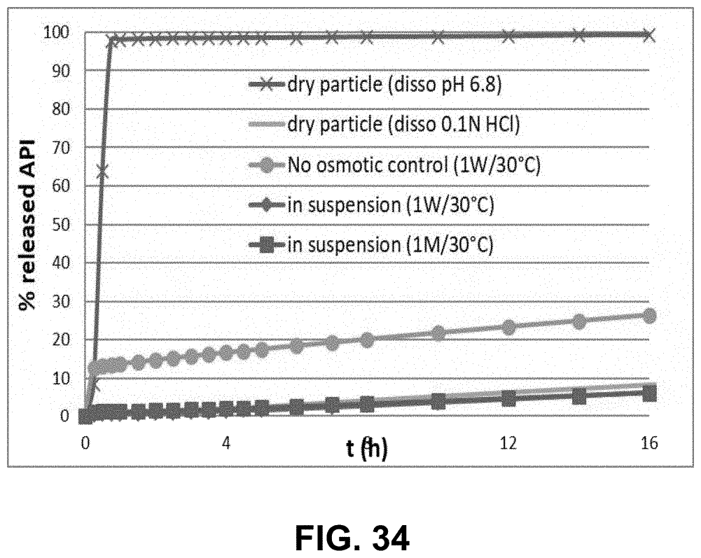

Several experiments were designed to test the stability of compositions comprising coated drug cores of acetaminophen (APAP), Metformin HCl or Dexmethylphenidate HCl, in a continuous phase under osmotic control. The coating weight ratio was 30 wt %. Drug-containing particles for a given drug were dispersed in a continuous phase (0.72 wt % of the total suspension for FIG. 33, 10 wt % of the total suspension for FIGS. 34-36), the continuous phase consisting of water and an osmotic agent and xanthan as a suspending agent (pH3).

Compositions were stored for various periods of time at 30° C., as indicated in Table 5. To evaluate the stability of the stored compositions, the in vitro dissolution profile of the stored composition was compared to the initial dissolution profile of the composition and/or the dry particle. Briefly, a sample volume of the composition was withdrawn from the container being stored after manual shaking of the container. Dissolution profiles were obtained by introducing a sample volume into a USP Type II apparatus equipped with 1 L vessels filled with 900 mL 0.1N HCl or phosphate buffer (PB) at pH 6.8. The vessels were maintained at 37° C. and the rotational speed of the paddles set at 100 RPM. The results are described in Table 5 and FIGS. 33-36. Overall, the compositions of exp. 15-19 had in vitro dissolution release profiles which remained stable upon storage for up to 1 month at 30° C.

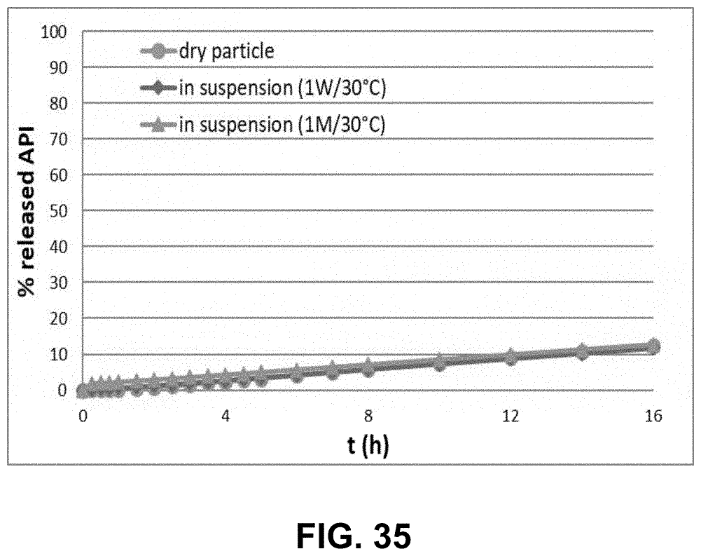

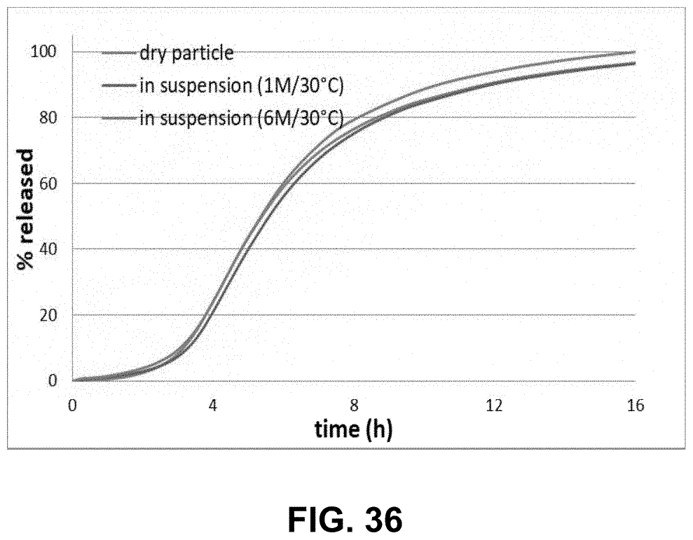

TABLE 5

| | | | | Samples |
|---|---|---|---|---|
| Exp. | Drug-containing particle | | Continuous | Dissolution Test |
| No. | Core | Coating | phase | Conclusions |
| 15 | 40 wt % CP203 | IR: CR22 | 85 wt % | Dry μP, composition stored for 1 w, 2 m or |
| | 54 wt % | 90% CA | Lycasin | 3 m @ 30° C. |
| | Dexmethylphenidate | 10% TEC | 1 wt % | USP II, 37° C., 0.1N HCl |
| | HCl | DR: CR20 | Avicel | The release profile of the composition is |
| | 6 wt % Klucel | 60% CAB | 0.3 wt % | stable upon storage for 3 months at 30° C. |
| | | 10% | xanthan | (FIG. 33). |
| | | EUDRAGIT ® | 0.1 wt % | |
| | | S100 | sodium | |
| | | 15% | benzoate | |
| | | EUDRAGIT ® | 0.6 wt % | |
| | | L100 | citric acid | |
| | | 15% CO | (pH 3) | |
| 16 | 20 wt % Cellet 127 | 40% | 60 wt % | Dry μP, composition stored for 1 w or 1 m |
| | 72 wt % APAP | EUDRAGIT ® | maltitol (in | @ 30° C. |
| | 8 wt % Klucel | L100 | suspension) | USP II, 37° C., 0.1N HCl |
| | | 60% CAB | 100% | USP II, 37° C., Phosphate 50 mM pH 6.8 |
| | | | water (no | The release profile of the composition is |
| | | | osmotic | stable upon storage for 1 month at 30° C. |
| | | | control) | under osmotic control (FIG. 34). |
| | | | (pH 3) | |
| 17 | 20 wt % Cellet 127 | 40% | 60 wt % | Dry μP, composition stored for 1 w or 1 m |
| | 72 wt % APAP | EUDRAGIT ® | maltitol | @ 30° C. |
| | 8 wt % Klucel | L100-55 | (pH 3) | USP II, 37° C., 0.1N HCl |
| | | 60% CAB | | The release profile of the composition is |
| | | | | stable upon storage for 1 month at 30° C. |
| | | | | (FIG. 35). |
| 18 | 20 wt % CP203 | 40% | 60 wt % | Dry μP, composition stored for 1 m or 6 m |
| | 65 wt % Metformin | EUDRAGIT ® | maltitol | @ 30° C. |
| | HCl | L100-55 | (pH 3) | USP II, 37° C., 0.1N HCl |
| | | 60% EC | | The release profile of the composition is |
| | | std100 | | stable upon storage for 6 months at 30° C. |
| | | | | (FIG. 36). |

1. A particle comprising a drug-containing core and an outer layer covering the core, wherein
the outer layer comprises a hydrophobic compound that is lipidic with a melting temperature between about 50° C. and about 90° C. and a gastro-soluble polymer comprising a minimum of 50% molar ratio of methyl methacrylate and a maximum of 50% molar ratio of an amino alkyl methacrylate, wherein the gastro-soluble polymer exhibits a Tg>50° C. and an overall Mw>50 000 g/mol, and wherein the hydrophobic compound and gastro-soluble polymer are present in a weight ratio of at least 2.3.

2. The particle of claim 1, wherein the hydrophobic compound and gastro-soluble polymer are present in a weight ratio of about 4.

3. The particle of claim 1, wherein the outer layer comprises about 70 wt % or more of the hydrophobic compound and about 30 wt % or less of the gastro-soluble polymer.

4. The particle of claim 3, wherein the outer layer comprises about 70 wt % to about 90 wt % of the hydrophobic compound and about 10 wt % to about 30 wt % or less of the gastro-soluble polymer.

5. The particle of claim 4, wherein the outer layer comprises about 75 wt % to about 85 wt % of the hydrophobic compound and about 15 wt % to about 25 wt % or less of the gastro-soluble polymer.

6. The particle of claim 1, wherein the outer layer consists about 70 wt % or more of the hydrophobic compound and about 30 wt % or less of the gastro-soluble polymer.

7. The particle of claim 6, wherein the outer layer consists of about 70 wt % to about 90 wt % of the hydrophobic compound and about 10 wt % to about 30 wt % or less of the gastro-soluble polymer.

8. The particle of claim 1, wherein the gastro-soluble polymer is a dimethyl amino ethyl methacrylate: methyl ethacrylate co-polymer.

9. The particle of claim 7, wherein the gastro-soluble polymer comprises an aqueous dispersion of a co-polymer comprising methyl methacrylate and diethylaminoethyl methacrylate in a ratio of about 6:4 (KOLLICOAT® SmartSeal 30 D).

10. The particle of claim 1, wherein the hydrophobic compound is selected from hydrogenated vegetable oils, vegetable waxes, wax yellow, wax white, wax microcrystalline, lanolin, anhydrous milk fat, hard fat suppository base, lauroyl macrogolglycerides, cetyl alcohols, polyglyceryl diisostearate, diesters of glycerol with at least one fatty acid, or triesters of glycerol with at least one fatty acid.

11. The particle of claim 10, wherein the hydrophobic compound has a Tₘ greater than or equal to about 50° C.

12. The particle of claim 10, wherein the hydrophobic compound comprises a plant-derived lubricant made from hydrogenated cottonseed oil (LUBRITAB®).

13. The particle of claim 1, wherein the particle further comprises one or more layers between the drug-containing core and the outer layer, optionally wherein one or more of the layers is a sustained-release layer or a delayed release layer.

14. The particle of claim 1, wherein the coating ratio of the outer layer is about 10 wt % to about 60 wt %.

15. The particle of claim 14, wherein the coating ratio is about 30 wt %.

16. The particle of claim 1, wherein the drug is 1,1-dimethylbiguanide hydrochloride (metformin hydrochloride), acetaminophen (APAP), or guaifenesin.

17. The particle of claim 1, wherein the drug-containing core comprises a drug-coated pellet, bead or resin.

18. The particle of claim 17, wherein the drug-containing core is a drug-coated pellet or bead.

19. The particle of any one of claim 1, wherein the drug-containing core comprises a crystalline form of the drug.

20. The particle of claim 1, wherein after storage of the particle for at least one month at about 30° C. as a suspension with a pH greater than 7.0 and an osmolality higher than the osmolality of a saturated solution of the drug in water and higher than a minimum value of 1300 mOsm/kg, the suspension has a stable in vitro dissolution profile.

21. The particle of claim 20, wherein the amount of drug in the continuous phase of the suspension is reduced by a factor of 2 compared to the saturation of the continuous phase by the drug.

22. The particle of claim 20, wherein the suspension has a stable in vitro dissolution profile and the amount of drug in the continuous phase of the suspension is reduced at least by a factor of 5 compared to the saturation of the continuous phase by the drug.

23. The particle of claim 20, wherein the suspension has a stable in vitro dissolution profile and the amount of drug in the continuous phase of the suspension is reduced at least by a factor of 10 compared to the saturation of the continuous phase by the drug.

24. A pharmaceutical composition comprising a plurality of particles dispersed in a continuous phase wherein:
the plurality of particles consist of particles according to any one of the preceding claims;
the continuous phase is (a) not saturated by the drug contained in the particles, (b) comprises at least one osmotic agent, and (c) has a pH that is above the gastro-soluble polymer's pKa; and
the osmolality of the continuous phase is (i) higher than saturated solution of the drug in water and (ii) higher than a minimum value of 1300 mOsm/kg.

25.-35. (canceled)

36. A pharmaceutical composition comprising a plurality of particles dispersed in a continuous phase wherein:
(a) each particle comprises a drug-containing core and an outer layer covering the core, wherein:
the drug has a coating/water partitioning coefficient K that is less than one; and
the outer layer comprises (i) up to 20 wt % of one or more water-soluble polymer and (ii) at least about 80 wt % of a mixture comprising a cellulosic derivative insoluble in the gastrointestinal tract and a plasticizer;
(b) the continuous phase is not saturated with a drug and comprises at least one osmotic agent;
(c) the osmolality of the continuous phase is higher than the osmolality of a saturated solution of the drug in water; and
(d) after storage of the composition in suspension for at least one month at about 30° C., the composition has a stable in vitro dissolution profile.

37.-60. (canceled)

61. A delayed-release pharmaceutical composition comprising a plurality of particles dispersed in a continuous phase wherein:
(a) each particle consists essentially of a drug-containing core and an outer layer covering the core, wherein the outer layer comprises one or more hydrophobic compounds that are crystalline in the solid state and one or more polymers carrying groups that are ionized at neutral pH, wherein the weight ratio of the hydrophobic compound(s) to the polymer(s) carrying groups that are ionized at neutral pH is greater than 1.5;
(b) the continuous phase (i) is not saturated with a drug, (ii) comprises at least one osmotic agent, and (iii) has a pH below 3.5;
(c) the osmolality of the continuous phase is higher than the osmolality of a saturated solution of the drug in water and greater than 1300 mOsm/kg; and
(d) after storage of the composition in suspension for at least one month at about 30° C., the composition has a stable in vitro dissolution profile.

62.-85. (canceled)
